(19) United States
(12) Patent Application Publication
(10) Pub. No.: US 2017/0335395 A1
(43) Pub. Date: Nov. 23, 2017

(54) A PARKINSON'S DISEASE DIAGNOSTIC BIOMARKER PANEL

(71) Applicant: THE TRUSTEES OF THE UNIVERSITY OF PENNSYLVANIA, Philadelphia (PA)

(72) Inventors: Alice CHEN-PLOTKIN, Philadelphia, PA (US); Benjamine LIU, Westlake Village, CA (US); Christine SWANSON, Silver Spring, MD (US)

(21) Appl. No.: 15/520,086

(22) PCT Filed: Oct. 26, 2015
(86) PCT No.: PCT/US15/57341

§ 371 Date: Apr. 19, 2017

Related U.S. Application Data
(60) Provisional application No. 62/069,078, filed on Oct. 27, 2014.

(51) Int. Cl. C12Q 1/68; G06F 19/24; G06F 17/18; G01N 33/68; G06F 19/12

(52) U.S. Cl. CPC C12Q 1/6883; G01N 33/6896; G06F 19/12; G06F 17/18; G06F 19/24; G01N 2800/2835; C12Q 2600/158; G01N 2800/60

(57) ABSTRACT

The present invention relates to a method of diagnosing Parkinson's disease in a subject using a novel set of biomarkers. The invention further includes compositions, methods and uses of a novel set of biomarkers to assess the risk of developing Parkinson's disease, to provide pre-symptomatic diagnosis of Parkinson's disease, and to assess prognosis of Parkinson's disease following therapeutic or other intervention.

### CROSS-REFERENCE TO RELATED APPLICATIONS

The present application claims priority under 35 U.S.C. §119(e) to U.S. Provisional Patent Application 62/069,078, filed Oct. 27, 2014, which application is incorporated herein by reference in its entirety.

### STATEMENT REGARDING FEDERALLY SPONSORED RESEARCH OR DEVELOPMENT

This invention was made with government support under grant number UO1-NS082134, awarded by the National Institutes of Health. The government has certain rights in the invention.

### BACKGROUND OF THE INVENTION

Parkinson's Disease is a progressive neurodegenerative disorder affecting more than 5 million people globally (Sherer, et al., 2011, Science Translational Medicine, 3, 79ps14). At a neuropathological level, Parkinson's disease is characterized by loss of dopaminergic neurons in the substantia nigra. However, two major diagnostic hurdles exist in the clinical management of Parkinson's disease patients. First, current medical practice for the diagnosis of Parkinson's disease relies almost entirely on clinical examination, with no laboratory-based confirmatory testing available. Clinical diagnosis is approximately 80% accurate in patients followed longitudinally with moderate symptoms (Hughes, et al., 1992, Journal of Neurology, Neurosurgery & Psychiatry, 55, 181-184). However, this accuracy may fall substantially, to approximately 65%, in the earlier stages of the disease (Rajput et al., 2014, Neurology, doi:10.1212/WNL.0000000000000653; Rajput, et al., 1991, Canadian Journal of Neurological Sciences, 18, 275-8). Second, by the time a Parkinson's disease patient becomes clinically symptomatic, it is estimated that 50% of substantia nigra dopaminergic neurons may already be lost (Fearnley, et al., 1991, Brain, 114, 2283-2301). These two facts together suggest that it is precisely in those patients in whom clinical diagnosis is difficult that a laboratory-based test has the greatest opportunity for therapeutic intervention. To date, however, no reliable laboratory-based test for the confirmation of Parkinson's disease, particularly in the early stages of the disease, exists.

Thus, there is a need in the art for a method of pre-symptomatic diagnosis of Parkinson's disease, a method of diagnosis of symptomatic Parkinson's disease, and also a method of assessing the risk of developing Parkinson's disease and of assessing prognosis of a treatment of Parkinson's disease though a laboratory-based test. The present invention meets this need.

### SUMMARY OF THE INVENTION

In one aspect, the present invention relates to a method of identifying a subject suspected of having Parkinson's disease for treatment thereof. Such method comprises determining the test level of a set of biomarkers in a sample obtained from the subject; and calculating the probability of the subject having Parkinson's disease according to Equation (I):

$$ln \frac{PD}{1 - PD} = A 1 - A 2 ( BDNF ) - A 3 ( Aminoacylase 1 ) - A 4 ( C 1 r ) + A 5 ( RAN ) + A 6 ( SRCN 1 ) + A 7 ( BSP ) + A 8 ( OMD ) - A 9 ( Growth hormone receptor ) - A 10 ( log Age ) - A 11 ( Gender ) . PD = probability of the subject having Parkinson ' s disease , \qquad ( I )$$

When the calculated probability is more than 0.5, then the subject is diagnosed with Parkinson's disease, and is administered at least one therapeutic compound selected from the group consisting of carbidopa-levodopa, a dopamine agonist, an MAO-B inhibitor, a catechol O-methyltransferase (COMT) inhibitor, an anticholinergic, and amantadine.

The determination of the test level of a set of biomarkers is conducted by a method selected from the group consisting of an antibody based assay, ELISA, western blotting, mass spectrometry, micro array, protein microarray, flow cytometry, immunofluorescence, PCR, aptamer-based assay, immunohistochemistry, and a multiplex detection assay.

The set of biomarkers comprises at least brain-derived neurotrophic factor (BDNF), aminoacylase-1, complement component 1 r subcomponent (C1r), Ras-related nuclear protein (RAN), SRC kinase signaling inhibitor 1 (SRCN1), bone sialoprotein (BSP), osteomodulin (OMD), and growth hormone receptor.

In certain embodiments, the variables in Equation (I) are as follows: A1=77.1531; A2=15.1212; A3=3.1383; A4=9.2111; A5=3.1337; A6=6.8268; A7=14.0165; A8=0.2854; A9=15.2483; A10=16.9700; and A11=1.8442.

In another aspect, the present invention includes a system of diagnosing Parkinson's disease using a non-transitory computer readable medium containing computer-readable program code including instructions for performing the diagnosis. The system comprises an assay determining the test level of a set of biomarkers, a computer hardware, and a software program stored in computer-readable media extracting the test level from the assay, calculating the probability of the subject having Parkinson's disease according to Equation (I), and outputting the result whether the subject having Parkinson's disease.

In yet another aspect, the present invention includes a kit for diagnosis of Parkinson's disease, the kit comprising testing reagents for a set of biomarker and an instructional material for use thereof.

In yet another aspect, the invention includes compositions, methods and uses of a novel set of biomarkers to assess the risk of developing Parkinson's disease, to provide a pre-symptomatic diagnosis of Parkinson's disease, and to assess prognosis of Parkinson's disease following therapeutic or other intervention. The set of biomarkers comprises at least brain-derived neurotrophic factor (BDNF), aminoacylase-1, complement component 1 r subcomponent (C1r), Ras-related nuclear protein (RAN), SRC kinase signaling inhibitor 1 (SRCN1), bone sialoprotein (BSP), osteomodulin (OMD), and growth hormone receptor.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1, comprising left, middle, and right panels, is a schematic diagram illustrating the overview of the study. Left panel illustrates the design of a classifier to discriminate Parkinson's disease from normal control (NC) samples with the training set and evaluation of its performance in the test set. Middle panel illustrates that all 96 Parkinson's disease and 45 NC samples in the combined training and test cohorts were used for further exploratory analyses. Right panel illustrates that the set of 90 proteins differentiating Parkinson's disease from NC within the combined cohort were used to assess the biological relevance of diagnostic biomarkers through functional pathway analysis.

FIG. 2 illustrates the process of identification of proteins that are differentially expressed in the plasma of Parkinson patients and normal control (NC) samples. Panel A is a Venn diagram illustrating proteins that were differentially expressed in the plasma of the 64 Parkinson's disease and 30 NC samples in the training set. Panel B is a table listing the top 30 proteins ranked by stability selection, along with the names of the proteins, Entrez names, P-values from the linear model, and directionalities (i.e., higher/lower concentration in Parkinson's disease compared to NC). Panel C is a scatterplot graph illustrating the distribution of proteins by direction and P-value for differentiating Parkinson's disease and NC.

FIG. 3 is a heatmap illustrating structure correlation between groups of proteins and individual samples.

FIG. 4 illustrates the process of developing a Parkinson's disease classifier test and its performance. Panel A is a diagram illustrating the stability selection process. Panel B is a graph illustrating Support Vector Machine (SVM) classifiers using the Radial Based Kernel built from training set data (n=64 Parkinson's disease, n=30 NC) using n=1 to n=30 of the top ranked biomarkers along with age and sex as covariates. Panel C is a graph illustrating a Logistic Regression classifier (red curve) built on training set data (n=64 Parkinson's disease, n=30 NC), using the top 8 proteins, with age and sex as covariates. Panel D is a graph illustrating use of the exact same eight-protein SVM and Logistic Regression classifiers developed in the training set to predict disease state in independent test set of 32 Parkinson's disease samples and 15 NC samples. Panel E is an equation illustrating how the exact Logistic Regression classifier equation is defined.

FIG. 5 illustrates tau, protease nexin 1, and brain-derived neurotrophic factor (BDNF) as plasma-based biomarkers. Panel A is a graph illustrating that tau is associated with earlier age at onset in Parkinson's disease. Panel B is a graph illustrating that protease nexin 1 is associated with earlier age at onset in Parkinson's disease. Panel C is a graph illustrating that BDNF measured by conventional immunoassay showed a moderately strong correlation with BDNF measured by the aptamer-based assay (Spearman correlation coefficient 0.62, p<0.001). Panel D is a graph illustrating quantification of plasma tau conducted using both the aptamer-based assay platform and a well-validated LUMINEX®-based immunoassay for CSF total tau to evaluate the aptamer-based assay's performance.

FIG. 6 illustrates the quality control measures for selecting proteins for PD diagnosis. Panel A is a Venn Diagram illustrating the number of proteins with a coefficient of variation (CV) greater than 20% among three quality control (QC) triplicates. Panel B is a Venn Diagram illustrating that two QC procedures were used to eliminate proteins from downstream analysis.

FIG. 7 illustrates pre-processing and normalization. Panel A illustrates normalization of sample data to eliminate intra-run hybridization variation using a set of hybridization reference standards introduced with sample eluate (“spiked-in”) on each array. Panel B illustrates hybridization normalization followed by median normalization to remove other potential assay biases within the run. Panel C is a graph illustrating the median test run using the set of 13 replicate calibrator samples. Panel D is a graph illustrating the tail test run using the set of 13 replicate calibrator samples.

FIG. 8 illustrates the reproducibility of Somalogic aptamer-based assay platform across space (UPenn vs. Boulder) and time (2013 vs. 2015). Left panel illustrates the reproducibility across all the proteins. Right panel illustrates the reproducibility across the top 94 proteins. Frequency distribution of individual protein assays with significant correlations (Spearman's Rho, with cutoff for significance indicated by vertical red dotted line) is shown.

### DETAILED DESCRIPTION OF THE INVENTION

The present invention includes compositions, methods and uses of a novel set of biomarkers to diagnose Parkinson's disease. The invention further includes compositions, methods and uses of a novel set of biomarkers to assess the risk of developing Parkinson's disease, to provide a pre-symptomatic diagnosis of Parkinson's disease, and to assess prognosis of Parkinson's disease following therapeutic or other intervention. Further, the present invention includes a method of detecting the biomarkers in a biological sample, and a kit useful in the practice of invention.

### Definitions

As used herein, each of the following terms has the meaning associated with it in this section.

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, and organic chemistry are those well-known and commonly employed in the art.

As used herein, the articles “a” and “an” refer to one or to more than one (i.e. to at least one) of the grammatical object of the article. By way of example, “an element” means one element or more than one element.

As used herein, the term “about” will be understood by persons of ordinary skill in the art and will vary to some extent on the context in which it is used. As used herein when referring to a measurable value such as an amount, a concentration, a temporal duration, and the like, the term “about” is meant to encompass variations of ±20% or ±10%, more preferably ±5%, even more preferably ±1%, and still more preferably ±0.1% from the specified value, as such variations are appropriate to perform the disclosed methods.

As used herein, the term “algorithm” refers to the equations and mathematical methods described herein used to calculate the probability of a subject having Parkinson's disease.

As used herein, the terms “comprising,” “including,” “containing” and “characterized by” are exchangeable, inclusive, open-ended and does not exclude additional, unrecited elements or method steps. Any recitation herein of the term “comprising,” particularly in a description of components of a composition or in a description of elements of a device, is understood to encompass those compositions and methods consisting essentially of and consisting of the recited components or elements.

As used herein, the term “consisting of” excludes any element, step, or ingredient not specified in the claim element.

As used herein, the term “consisting essentially of” does not exclude materials or steps that do not materially affect the basic and novel characteristics of the claim. “Instructional material,” as that term is used herein, includes a publication, a recording, a diagram, or any other medium of expression that can be used to communicate the usefulness of the composition and/or compound of the invention in a kit. The instructional material of the kit may, for example, be affixed to a container that contains the compound and/or composition of the invention or be shipped together with a container that contains the compound and/or composition. Alternatively, the instructional material may be shipped separately from the container with the intention that the recipient uses the instructional material and the compound cooperatively. Delivery of the instructional material may be, for example, by physical delivery of the publication or other medium of expression communicating the usefulness of the kit, or may alternatively be achieved by electronic transmission, for example by means of a computer, such as by electronic mail, or download from a website.

The term “pre-symptomatic diagnosis” refers to a diagnosis of Parkinson's disease before the manifestation of clinical motor symptoms such as bradykinesia, rigidity, tremor, and postural instability that would ordinarily lead to clinical diagnosis.

As used herein, a “subject” may be a human or non-human mammal or a bird. Non-human mammals include, for example, livestock and pets, such as ovine, bovine, porcine, canine, feline and murine mammals. Preferably, the subject is human.

As used herein, the term “test level” refers to the level of a set of biomarkers in a biological sample from a subject who will be evaluated as to whether the subject may have Parkinson disease or is at risk of developing Parkinson disease.

Throughout this disclosure, various aspects of the invention may be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range and, when appropriate, partial integers of the numerical values within ranges. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

In one aspect, the present invention includes an in vitro method for diagnosis of Parkinson's disease by measuring a set of biomarkers in a sample obtained from a subject. The method comprises obtaining a biological sample from a subject; detecting the test level of a set of biomarkers in the sample; calculating the probability of the subject having Parkinson's disease according to Equation (I); when the calculated probability is more than 0.5, then the subject is diagnosed with Parkinson's disease and treatment may be initiated. Also, the calculated probability can be used for risk assessment, pre-symptomatic diagnosis, or prognosis.

$$ln \frac{PD}{1 - PD} = A 1 - A 2 ( BDNF ) - A 3 ( Aminoacylase 1 ) - A 4 ( C 1 r ) + A 5 ( RAN ) + A 6 ( SRCN 1 ) + A 7 ( BSP ) + A 8 ( OMD ) - A 9 ( Growth hormone receptor ) - A 10 ( log Age ) - A 11 ( Gender ) ; PD = probability of the subject having Parkinson ' s disease \qquad ( I )$$

In equation (I), units are in Relative Fluorescence Units (RFUs) as measured on an aptamer-based platform (SOMASCAN assay) produced by Somalogic, Inc. These RFUs are convertible to customary mg/mL concentration space via loading of samples with known concentration to generate a standard curve. Variable Age is the age of the subject in years; function log is the logarithm with base 10; variable Gender can have values of 1 or 0 with Males=1 and Females=0.

In certain embodiments, A1 is a constant within the range of 70 to 80; A2 is a coefficient factor of brain-derived neurotrophic factor (BDNF) within the range of 0 to 20; A3 is a coefficient factor of aminoacylase-lwithin the range of 0 to 5; A4 is a coefficient factor of complement component 1 r subcomponent (C1r) within the range of 0 to 10; A5 is a coefficient factor of Ras-related nuclear protein (RAN) within the range of 0 to 5; A6 is a coefficient factor of SRC kinase signaling inhibitor 1 (SRCN1) within the range of 0 to 8; A7 is a coefficient factor of bone sialoprotein (BSP) within the range of 0 to 16; A8 is a coefficient factor of osteomodulin (OMD) within the range of 0 to 1; A9 is a coefficient factor of growth hormone receptor within the range of 0 to 18; A10 is a coefficient factor of log Age with the range of 0 to 18; A11 is a coefficient factor of Gender with the range of 0 to 3.

In one embodiment, A1=77.1531; A2=15.1212; A3=3.1383; A4=9.2111; A5=3.1337; A6=6.8268; A7=14.0165; A8=0.2854; A9=15.2483; A10=16.9700; A11=1.8442; and Equation (I) becomes Equation (II).

$$ln \frac{PD}{1 - PD} = 77.1531 - 15.1212 ( BDNF ) - 3.1383 ( Aminoacylase 1 ) - 9.21111 ( C 1 r ) + 3.1337 ( RAN ) + 6.8268 ( SRCN 1 ) + 14.0165 ( BSP ) + 0.2854 ( OMD ) - 15.2483 ( Growth hormone receptor ) - 16.9700 ( log Age ) - 1.8442 ( Gender ) ; PD = probability of the subject having Parkinson ' s disease \qquad ( II )$$

The methods described herein can be readily implemented in software that can be stored in computer-readable media for execution by a computer processor. For example, the computer-readable media can be volatile memory (e.g., random access memory and the like) and/or non-volatile memory (e.g., read-only memory, hard disks, floppy disks, magnetic tape, optical discs, paper tape, punch cards, and the like).

Additionally or alternatively, the methods described herein can be implemented in computer hardware such as an application-specific integrated circuit (ASIC).

Additionally or alternatively, the methods described herein can be also readily implemented in a system comprising an assay determining the test level of a set of biomarkers described herein; a computer hardware; and a software program stored in computer-readable media extracting the test level from the assay; calculating the probability of the subject having Parkinson's disease according to Equation (I) and outputting the result whether the subject having Parkinson's disease.

In another aspect, the set of biomarkers described herein is anticipated to be used for pre-symptomatic diagnosis of Parkinson's disease; risk assessment of development of Parkinson's disease; and evaluation of the prognosis of treatments for Parkinson's disease.

### Biomarker

A “biomarker” is any gene, protein, or metabolite whose level of expression in a tissue, cell or bodily fluid is dysregulated compared to that of a normal or healthy cell, tissue, or biological fluid. Biomarkers to be measured in the methods of the invention are selectively altered when a subject has developed, or is at risk of developing Parkinson's disease.

Currently, no biochemical tests measuring a set of biomarkers as confirmation of Parkinson's disease are available. Instead, diagnosis of Parkinson's disease relies almost entirely on the physician's history and clinical exam, with an estimated accuracy of <80% (Hughes, et al., 1992, Neurology, 42, 1142-1142). Thus, the development of a reliable assay for diagnostic confirmation of Parkinson's disease is useful in both the clinical care of Parkinson's disease patients and in subject selection for clinical trials for potential Parkinson's disease-modifying drugs.

In one embodiment, the biomarkers are proteins. In another embodiment, the biomarkers are mRNA or DNA, encoding these proteins.

In one embodiment, the set of biomarkers disclosed in the present invention comprises brain-derived neurotrophic factor (BDNF), aminoacylase-1, complement component 1r subcomponent (C1r), Ras-related nuclear protein (RAN), SRC kinase signaling inhibitor 1 (SRCN1), bone sialoprotein (BSP), osteomodulin (OMD), and growth hormone receptor.

In another embodiment, the set of biomarkers useful in the present invention comprises mRNA or DNA encoding brain-derived neurotrophic factor (BDNF), aminoacylase-1, complement component 1 r subcomponent (C1r), Ras-related nuclear protein (RAN), SRC kinase signaling inhibitor 1 (SRCN1), bone sialoprotein (BSP), osteomodulin (OMD), and growth hormone receptor.

Brain-derived neurotrophic factor (BDNF) is a secreted protein encoded by BDNF gene. BDNF acts on certain neurons of the central nervous system and the peripheral nervous system, supporting existing neurons and helping the growth and differentiation of new neurons and synapses (Acheson, et al., 1995, Nature, 374: 450-3; Huang, et al., 2001, Annu. Rev. Neurosci., 24: 677-736).

Aminoacylase-1 is a zinc binding enzyme which hydrolyzes N-acetyl amino acids into the free amino acid and acetic acid. It is encode by Aminoacylase-1 gene located on the short arm of chromosome 3 (Miller, et al., 1991, Genomics, 8 (1): 149-154; Voss, et al., 1982, Ann Hum Genet 44 (Pt 1): 1-9).

Complement component 1 r subcomponent (C1r) is a protein involved in the complement system. It catalyzes cleavage of Lys(or Arg)-Ile bond in complement subcomponent C1s to form C verbar 1s (Sim, et al., 1986, Biochemistry, 25: 4855-4863). C1R is a secreted protein belonging to the peptidase S1 family. The mature protein extends from residues 18-705, after cleavage of the signal peptide extending from 1-17. C1R is further cleaved into two peptides: complement Clr subcomponent light chain (residues 18-462) and heavy chain (residues 464-705).

Ras-related nuclear protein (RAN) is a protein encoded by the RAN gene, also known as GTP-binding nuclear protein Ran. Ran is a small 25 kDa protein that is involved in transport into and out of the cell nucleus during interphase and also involved in mitosis. It is a member of the Ras superfamily (Moore, et al., 1994, Trends Biochem. Sci., 19 (5): 211-6; Dasso, et al., 1998, Am. J. Hum. Genet., 63 (2): 311-6; Avis, et al., 1996, J. Cell. Sci., 109 (10): 2423-7).

SRC kinase signaling inhibitor 1 (SRCN1) is a protein, involved in calcium-dependent exocytosis and may play a role in neurotransmitter release or synapse maintenance (Ito, et al., 2008, J. neurochem, 107, 61-72; Chin, et al., 2000, J. Biol, Chem. 275, 1191-200).

Bone sialoprotein (BSP) is a protein and a component of mineralized tissues such as bone, dentin, cementum and calcified cartilage. It was originally isolated from bovine cortical bone as a 23-kDa glycopeptide with high content (Williams, et al., 1965, Biochim. Biophys. Acta, 101 (3): 327-35; Herring, et al., 1964, Nature, 201 (4920): 709).

Osteomodulin (OMD) is a protein encoded by the OMD gene (Maruyama, et al., 1994, Gene, 138 (1-2): 171-4).

Growth hormone receptor is a protein encoded by the growth hormone receptor gene. Binding growth hormone to the receptor leads to receptor dimerization and the activation of an intra- and intercellular signal transduction pathway leading to growth (Gonzalez, et al., 2007, Growth Horm IGF Res., 17(2): 104-112).

### Methods for Detecting

Detection of a protein, an mRNA or a DNA is well known in the art. The detecting methods described herein are exemplary and should not be construed as limiting the invention in any way. Non-limiting examples for detecting a protein or an mRNA or a DNA include an antibody based assays, ELISA, western blotting, mass spectrometry, protein microarray, PCR, aptamer-based assay, SOMASCAN® assay, LUMINEX®-based immunoassay and a multiplex detection assay.

Immunoassay

An immunoassay is a biochemical test that measures the amount of a macromolecule in a sample through use of antibody or immunoglobulin. The macromolecule, referred to as an analyte detected by the immunoassay is in many cases a protein. Analytes in biological liquids such as serum or urine are frequently measured using immunoassays for medical and research purposes (Yetisen, et al., 2013, Lab on a Chip, 13 (12): 2210-2251).

Immunoassays are available in many different formats and variations, all of which are should be construed to be included in the present invention Immunoassays may be run in multiple steps or a single step. Multi-step assays are often called separation immunoassays or heterogeneous immunoassays. Some immunoassays are conducted simply by mixing the reagents and sample and making a physical measurement. Such assays are called homogenous immunoassays (Shah et al., 1992, Pharm. Res., 9(4): 588-592; Desilva et al., 2003, Pharm. Res., 20 (11): 1885-1900; Swartzman, et al., 1999, Analytical Biochemistry, 271: 143-151).

ELISA

The enzyme-linked immunosorbent assay (ELISA) is one kind of immunoassy and is a test that uses antibodies and color change to identify a substance. There are many types of ELISA, including indirect ELISA (Koenig, et al, 1981, Journal of General Virology, 55: 53-62), sandwich ELISA (Tomoyuki, et al., 1996, British J. of Haematology, 93: 783-788), competitive ELISA (EP0202890 A2), and multiple and ready to use ELISA (US20140154257 A1).

Western Blotting

The western blot, also referred to the protein immunoblot, is a widely used analytical technique used to detect specific proteins in a sample of tissue homogenate or extract. It uses gel electrophoresis to separate native proteins by 3-D structure or denatured proteins by the length of the polypeptide. The proteins are then transferred to a membrane, where they are stained with antibodies specific to the target protein (Towbin, et al., 1979, Proceedings of the National Academy of Sciences USA, 76 (9): 4350-54; Renart, et al., 1979, Proceedings of the National Academy of Sciences USA, 76 (7): 3116-20).

Mass Spectrometry

Mass spectrometry (MS) is an analytical chemistry technique that measures the mass-to-charge ratio and abundance of gas-phase ions. The two primary methods for ionization of whole proteins are electrospray ionization (ESI) and matrix-assisted laser desorption/ionization (MALDI). In top-down strategy of protein analysis, intact proteins are ionized by either of the two techniques described above, and then introduced to a mass analyzer. In a break-down strategy of protein analysis, proteins are enzymatically digested into smaller peptides using proteases such as trypsin or pepsin, either in solution or in gel after electrophoretic separation.

One method of quantitation of proteins by mass spectrometry involves heavier isotopes of carbon (¹³C) or nitrogen (¹⁵N) and light isotopes (e.g. ¹²C and ¹⁴N) (Snijders, et al., 2005, J. Proteome Res., 4 (2): 578-85). The most popular methods for isotope labeling are SILAC (stable isotope labeling by amino acids in cell culture), trypsin-catalyzed ¹⁸O labeling, ICAT (isotope coded affinity tagging), and iTRAQ (isobaric tags for relative and absolute quantitation) (Miyagi, et al, 2007, Mass Spectrometry Reviews, 26 (1): 121-136.)

Protein Microarray

A protein microarray (or protein chip) is a high-throughput method used to track the interactions and activities of proteins on a large scale. Its advantage lies in the fact that large numbers of proteins can be tracked in parallel. Probe molecules, labeled with a fluorescent dye, are added to the array of protein.

Multiplex Detection Assay

A multiplex detection assay is a type of assay that simultaneously measures multiple analytes (dozens or more) in a single run/cycle of the assay. It is distinguished from procedures that measure one analyte at a time. A multiplex detection assay for nucleic acid detection includes DNA microarray, SAGE, multiplex PCR, multplex ligation-dependent proble amplification and LUMINEX®/XMAP®. A multiplex detection assay for protein detection includes protein microarray, antibody microarray, phage display, and LUMINEX®/XMAP®.

Aptamer-Based Assay

Aptamer-based assay is an assay based on aptamers' high affinity and specificity towards a wide range of target molecules. Aptamers are single stranded DNA or RNA oligonucleotides with low molecular weight, amenable to chemical modifications and exhibit stability undeterred by repetitive denaturation and renaturation (Citartan, et al., 2012, Biosensors and Bioelectronics, 34:1, 1-11; Qureshi, et al., Biosensors and Bioelectronics, 34:1, 165-170).

SOMASCAN® Assay

SOMASCAN® assay is a proprietary aptamer-based assay used to simultaneously measure thousands of proteins from small sample volumes (Gold, et al., 2012, New Biotechnology, 29, 543-549). It was developed by Somalogic Inc. (Boulder, USA).

LUMINEX®-Based Immunoassay

LUMINEX®-based immunoassay is a proprietary multiplex bead-based immunoassay testing platform simultaneously measures multiple analytes by exciting a sample with a laser, and subsequently analyzing the wavelength of emitted light (Haasnoot, et al., 2007, J. Agric. Food Chem., 55 (10), 3771-3777; Anderson, et al., Environ. Sci. Technol., 2007, 41 (8), pp 2888-2893; Liu, et al., 2005, Clinical Chemistry, 51:7, 1102-1109). It was developed by Luminex Inc. (Austin, US).

Biological Sample

The biological sample described herein may be urine, blood or cerebrospinal fluid. Blood includes whole blood, blood plasma, and blood serum. In one embodiment, the biological sample is blood plasma. In another embodiment, the biological sample is cerebrospinal fluid.

### Treatment:

The appropriate therapeutic intervention for treatment of Parkinson's disease is generally administration of one or more compositions to a subject suffering from Parkinson's disease. Such compositions may be selected from the group consisting of carbidopa-levodopa, dopamine agonists, monoamine oxidase B (MAO-B) inhibitors, catechol O-methyltransferase (COMT) inhibitors, anticholinergics, and amantadine.

In one embodiment, the treatment is administration of levodopa to the subject. In another embodiment, dopamine agonists, including but not limited to pramipexole, ropinirole, rotigotine and apomorphine, are administered. In yet another embodiment, MAO-B inhibitors, including but not limited to selegiline and rasagiline, are administered. In yet another embodiment, COMT inhibitors, including but not limited to entacapone and tolcapone, are administered. In yet another embodiment, anticholinergics, including but not limited to benztropine and trihexyphenidyl, are administered. In yet another embodiment, amantadine is administered.

### Kit

The present invention also includes a kit. The kit comprises reagents to detect and quantify the set of biomarker described elsewhere herein, and instruction material for using the kit. In one embodiment, the kit is useful for diagnosis of Parkinson's disease; in another embodiment, the kit is useful for pre-symptomatic diagnosis of Parkinson's disease; in yet another embodiment, the kit is useful for risk assessment of development of Parkinson's disease; in yet another embodiment, the kit is useful for evaluation of the prognosis of treatments for Parkinson's disease.

### Examples

The invention is now described with reference to the following Examples. These Examples are provided for the purpose of illustration only, and the invention is not limited to these Examples, but rather encompasses all variations that are evident as a result of the teachings provided herein.

### Materials and Methods

### Overview of Study Design

A total of 97 Parkinson's disease and 45 normal control (NC) subjects were assessed for the presence of and concentrations of 968 plasma proteins in their plasma. Two-thirds (n=95) of these subjects were pre-allocated to a training set, and one-third (n=47) of the subjects were allocated to a test set, in order to develop a panel of classifying proteins for Parkinson's disease diagnosis. Enrollment criteria are described in the Subjects section below. All data points were included with the exception of one Parkinson's disease sample. This sample was found to have a median normalization scale factor higher than the pre-specified expected range during data pre-processing and was therefore eliminated as an outlier.

Validation of the biomarkers was achieved in two ways. First, within the 94-sample training set, proteins were selected for classifier inclusion through 100,000 re-samples of the data (jack-knifed with 10% samples and 30% proteins removed randomly), and classifier performance was assessed through 10-fold cross-validation. These measures assess the robustness of the proteins selected and the classifier that was built, but they do not represent an independent replication in the strictest sense. Therefore, classifier performance was assessed in the 47-sample independent test set, which was not used for selection of proteins that should be included in the classifier or for building the classifier itself.

### Subjects

Clinical data and plasma samples were collected from 97 Parkinson's disease patients and 45 normal controls (NC) at the University of Pennsylvania (UPenn) enrolled for research under IRB approval. One Parkinson's disease sample was found to have outlier values in pre-processing and normalization steps on the aptamer-based assay; thus 96 Parkinson's disease samples yielded the plasma protein measurements disclosed herein. All 45 NC samples gave rise to acceptable plasma protein measurements. Clinical and demographic details of all subjects having plasma samples used in the study are presented in Table 1. In addition, for tau assay validation purposes, cerebrospinal fluid (CSF) was collected from 80 research subjects at UPenn under IRB approval; clinical and demographic details for these 80 subjects are presented in Table 2. All Parkinson's disease patients met the diagnostic criteria of the United Kingdom Parkinson's Disease Brain Bank and were part of a longitudinal, extensively characterized cohort at UPenn. In order to control for environmental biases, age and sex were matched between Parkinson's disease and control groups, and NCs were recruited primarily from the unaffected spouses of Parkinson's disease cases from the same clinic.

### Plasma Collection

Plasma samples from both Parkinson's disease and NC groups were collected, processed, and stored in parallel. Plasma samples were collected according to IRB-approved protocols as previously described (Chen-Plotkin, et al., 2011, Annals of Neurology, 69, 655-663). Plasma was obtained using EDTA tubes (BD Vacutainer, Franklin Lanes, N.J., USA). Samples were then immediately put on ice and centrifuged at 3000 rpm×15 min at 4° C. 0.5 mL aliquots were created in polypropylene 2 mL cryovials (Corning cryovials, Acton, Mass., USA) for storage at −80° C. until use.

### Protein Quantification

All samples were assayed together, where the operators of the test were blinded as to disease status. Proteins were quantified using SOMASCAN®, an aptamer-based technology from SomaLogic Inc. (Boulder, Colo.) (Gold, et al., 2010, PLoS ONE, 5, e15004). This proteomics platform is made possible by protein-capture SOMAMER® (Slow Off-rate Modified Aptamer), chemically modified oligonucleotides with specific affinity to their protein targets, developed by in vitro selection (SELEX®) as previously described (Davies, et al., 2012, Proceedings of the National Academy of Sciences, doi:10.1073/pnas.1213933109; Dewey, et al., 1995, J. Am. Chem. Soc. 117, 8474-8475).

The specific steps of the SOMASCAN® assay have been previously outlined and described in detail in technical white papers at www.somalogic.com. In brief, plasma samples are incubated with the reagent mixes containing SOMAMER® to 1129 different proteins to allow for equilibrium binding of fluorophore-tagged SOMAMER® to their protein targets. Next, a series of partitioning and wash steps are used to capture only the SOMAMER® that are bound to their cognate proteins. Finally, the protein-bound SOMAMER® oligonucleotides are released from the protein complex, captured by complementarity, and quantified using DNA hybridization arrays.

To adjust for batch-to-batch variation, the hybridization arrays are normalized and calibrated using data from a reference set of pooled plasma samples run on each batch. Thus, the normalized and calibrated signal for each SOMAMER®—reported in relative fluorescence units (RFU)—reflects the relative amount of each cognate protein present in the original sample. Raw SOMALOGIC® data (in RFUs) was log-transformed prior to analysis.

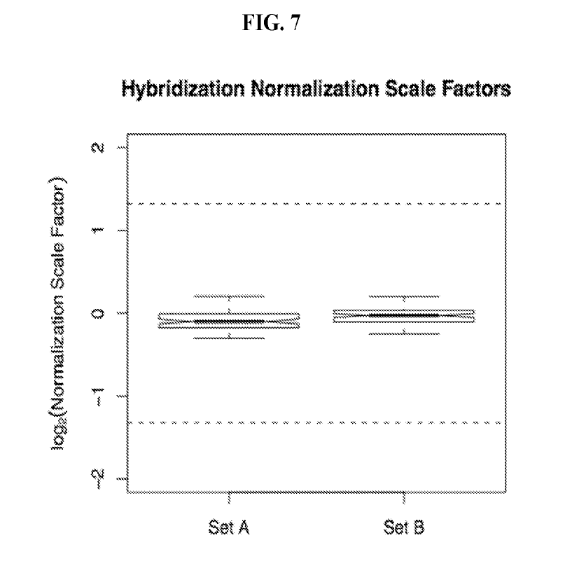

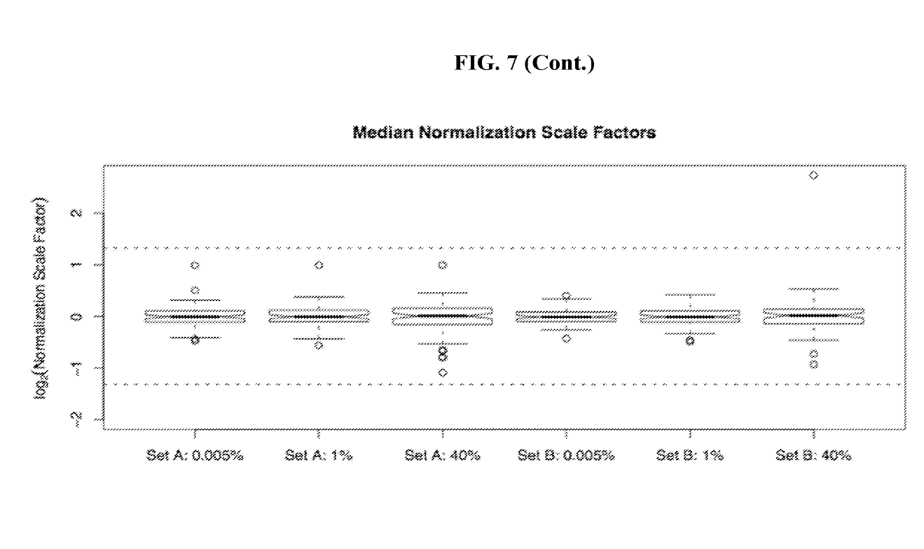

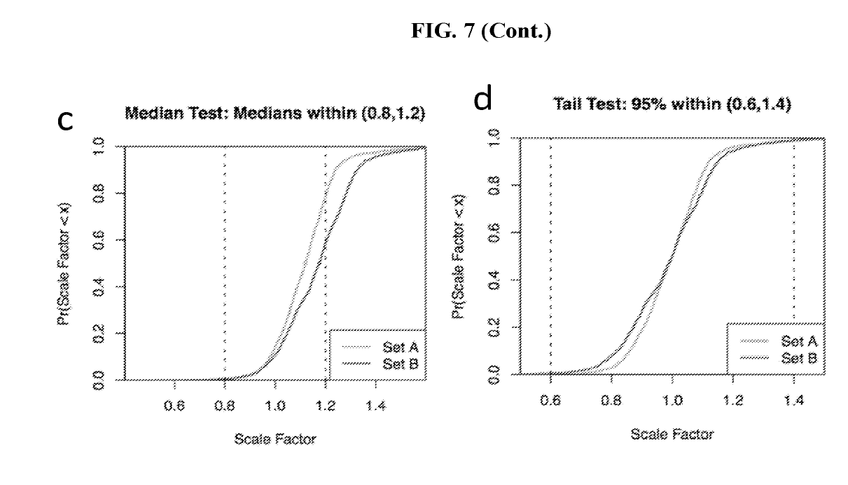

Plasma samples for the present study were assayed in two sets (Set A and Set B) using Version 3.0 of the SOMASCAN® assay, along with hybridization standards, 13 plasma calibrator samples, and 2 buffer (no protein) control samples. Sample data were normalized to eliminate intra-run hybridization variation using a set of hybridization reference standards introduced with sample eluate (“spiked-in”) on each array (FIG. 7, Panel A). Scaling factors to normalize for hybridization variability are shown. Medians, interquartile ranges (IQR), and full ranges for scaling factors are shown as box (IQR) and whiskers (full range) plots for each set of samples, demonstrating minimal hybridization variability. Hybridization normalization was followed by median normalization to remove other potential assay biases within the run (FIG. 7, Panel B). The expectation is that for each individual sample, the median value across all proteins will be reasonably close, even though individual proteins may differ across individuals or groups. Scaling factors for median normalization are shown as box (IQR) and whiskers (full range) plots for each set of samples, at three different sample dilutions. Only one Parkinson's disease sample had median normalization scale factors outside the acceptable range (indicated by dashed lines) and was removed from the downstream analysis (Set B, at the 40% dilution).

Following hybridization and median normalization, the data were then calibrated to remove assay differences between runs using the set of 13 replicate calibrator samples. For each SOMAMER®, the median value across the 13 replicate calibrator samples in the run was determined. This value was then compared to an outside reference value for the calibrators obtained across many runs in many experiments, and a scaling factor (ratio of reference calibrator value to median observed calibrator value) for calibration normalization was obtained. This resulting scale factor was then applied to empirical data to reduce inter-run variation. In this study, both runs passed the acceptance criteria, with scale factors between 0.8-1.2 for the majority of samples (FIG. 7, Panel C) and 95% of the SOMAMER® having calibration scale factors between 0.6 and 1.4 (FIG. 7, Panel D).

### Quality Control of Proteins

Three sets of triplicate identical aliquots (a total of nine samples) were placed at random within two large batches of samples quantified in the SOMASCAN® assay. These 9 quality control (QC) samples were separated in time so that they could capture both temporal and reagent batch variability. The coefficients of variation (CVs) were calculated from these 3 sets of triplicate QC samples (i.e. for each protein three CVs were calculated) using the raw SOMALOGIC® data (in RFUs). Proteins that had CVs greater than 0.2 from any one of the triplicates were eliminated in the downstream analysis. Moreover, proteins with more than 25% of measurements in the sample set below the lower limits of detection (LLOD) or above the upper limits of detection (ULOD) were further eliminated from dataset for the plasma assay, as imputation of missing values constituting more than 25% of the total sample set becomes relatively unstable (Scheffer, et al., 2002, Res. Lett. Inf. Math. Sci., 3, 153-160).

### Statistical Methods

### Single Protein Analysis

For all proteins passing the QC tests described above, Mann-Whitney U-tests (non-parametric), Student's t-tests (parametric), and permutation tests (10,000 resamples) were performed to find proteins differentially expressed in training set of 64 Parkinson's disease versus 30 NC subjects. A nominal p-value cutoff of 0.005 was employed for all three tests. All proteins were screened to pass QC tests for those whose plasma concentration associated significantly with disease category (Parkinson's disease versus NC) in a linear model co-varying for the levodopa equivalent daily dose (LEDD), age at plasma collection, and sex. A nominal p-value cutoff of 0.005 was employed for associations between candidate proteins and disease category in this linear model. Using the intersection of those proteins found to differentiate Parkinson's disease versus NC by all methods (Mann-Whitney U test, Student's t-test, permutation tests, and linear model), candidate diagnostic biomarkers for Parkinson's disease were nominated for downstream analyses.

### Hierarchical Clustering and Heatmap Generation

Heatmaps were generated using the PARTEK® GENOMICS SUITE® version 6.6. Raw SOMALOGIC® data (in RFUs) was log-transformed, and these values were standardized by setting each protein to a mean of zero and standard deviation of 1. Both individual subjects and proteins were then hierarchically clustered by Euclidean distance using the average linkage.

### Stability Selection Ranking

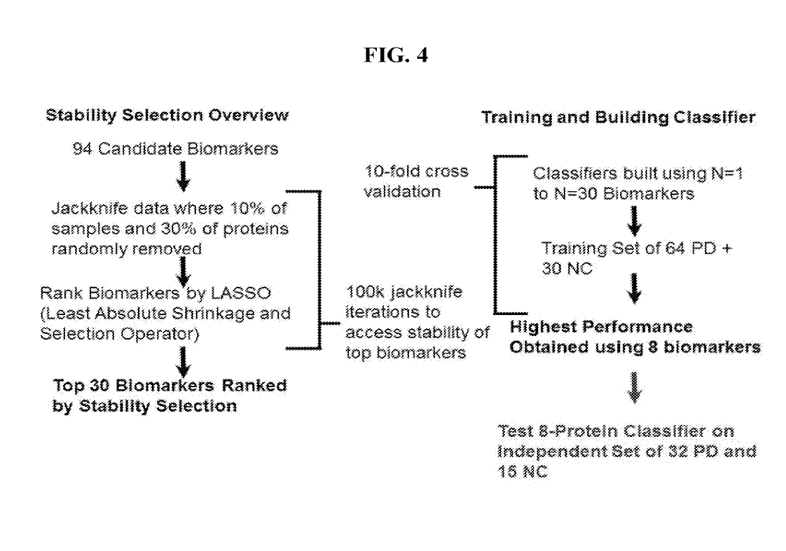

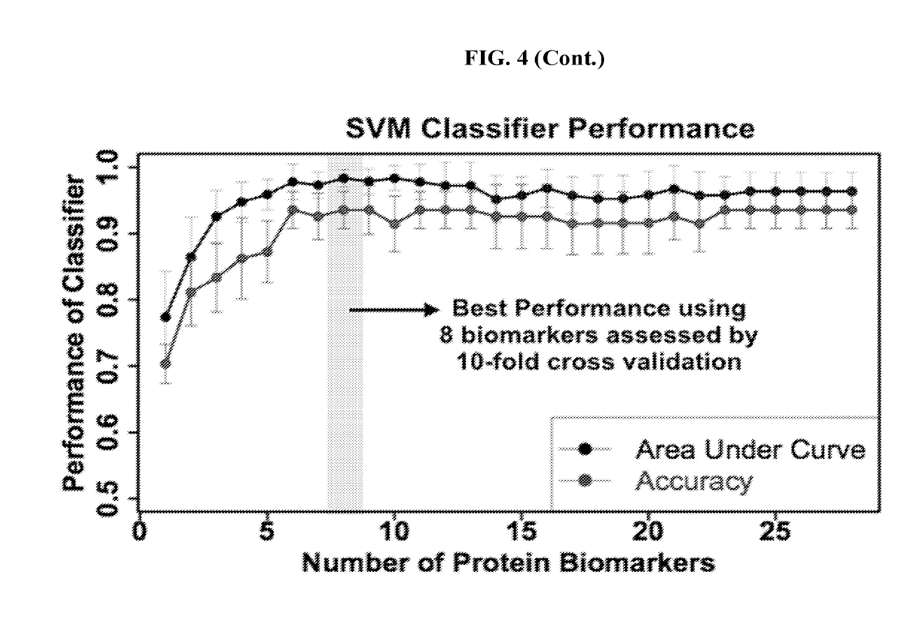

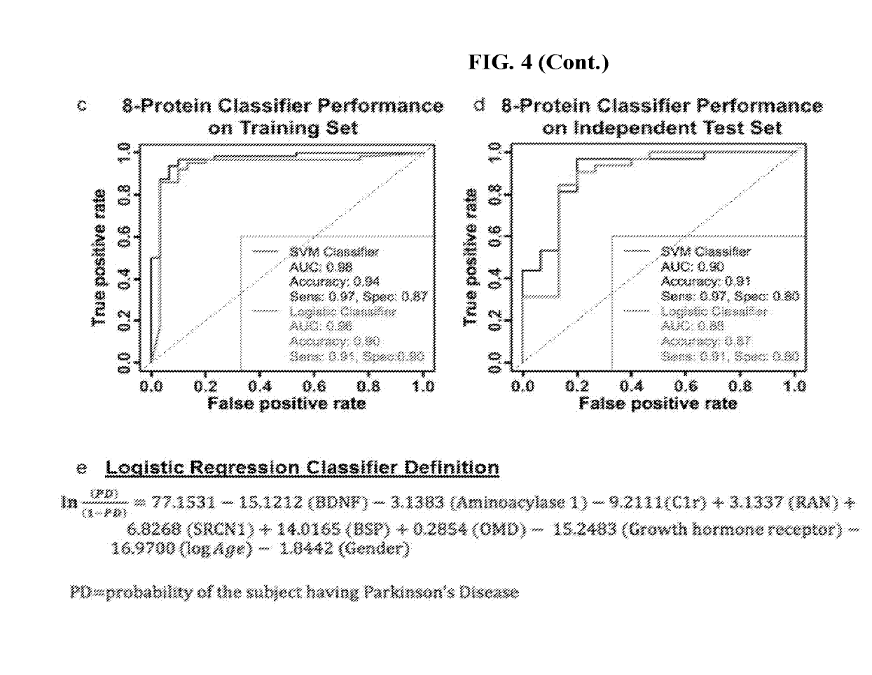

Stability selection is a meta-statistical tool that identifies consistently important features by repeated sub-sampling of the data (Meinshausen, et al., 2010, Journal of the Royal Statistical Society: Series B (Statistical Methodology), 72, 417-473). Stability selection using the Least Absolute Shrinkage and Selection Operator (LASSO) method (Tibshirani, et al., 1996, Journal of the Royal Statistical Society: Series B (Statistical Methodology), 58, 267-288) was implemented to rank candidate biomarkers using the BIOMARK® package in R across 100,000 jackknifed iterations (Wehrens, et al., 2011, Analytica Chimica Acta, 705, 15-23). At each iteration, 30% of the proteins and 10% of the samples were removed from the 94-sample (64 Parkinson's disease, 30 NC) training set, and LASSO was used to feature-select for variables on the remaining data. Proteins were ranked by the proportion of iterations in which they were found to have a non-zero coefficient using LASSO (FIG. 4, Panel A). R-scripts for these analyses are available upon request.

### Construction and Evaluation of Diagnostic Classifiers

Classifiers were built in two ways, using Support Vector Machines (Radial Based Kernel) and Logistic Regression, applied to data from the 94-sample (64 Parkinson's disease, 30 NC) training set. The optimal number of biomarkers was determined to use in the diagnostic panel by creating classifiers using n=1 to n=30 of the top-ranked biomarkers, in addition to age and sex as input features (FIG. 4, Panel B). Model parameters for C and γ for the Support Vector Machine classifiers were determined using a grid search where the C parameter ranged from 1 to 100 by intervals of 10 and γ ranged from 0.001 to 0.01 by intervals of 0.001. The model parameters of C=11 and γ=0.005 were used for SVM classifier.

Classifiers were assessed using 10-fold cross-validation within the training set samples. The optimum number of proteins to include in the classifier was assessed by AUC and accuracy, adjusting for age at plasma sampling and sex. ROC curves were drawn using the ROCR package in R (FIG. 4, Panel C) (Sing, et al., 2005, Bioinformatics, 21, 3940-3941). Support vector machines were built using the e1071 package in R and Logistic Regression classifiers were built using the glm( ) function in R (Hsu, et al, 2002, Trans. Neur. Netw., 13, 415-425).

Having constructed two different diagnostic classifiers (SVM and Logistic Regression classifiers) as described above using the training set, the ability of these exact classifiers to predict disease state were evaluated in an independent test set of 32 Parkinson's disease and 15 NC subjects. Of note, test set samples were never used in the analysis stream leading to the construction of the classifiers. ROC curves were drawn using the ROCR package in R (FIG. 4, Panel D) (Sing, et al., 2005, Bioinformatics, 21, 3940-3941).

Classifier construction and evaluation were performed in R, and R-scripts are available upon request.

### Biological Pathway Analysis

The strict partitioning of samples into a training and test set is important in assessing classifier performance. However, for exploratory analyses aimed at understanding disease pathophysiology, the training and test sets were combined to maximize information. Thus, a combined set of 141 samples (96 Parkinson's disease and 45 NC) was formed and applied the same methodology previously used in the training set alone to nominate candidate biomarkers from this combined sample set. In brief, Mann-Whitney U tests, Student's t-tests, permutation tests (10,000 resamples) and the linear regression model were used to nominate candidate Parkinson's disease biomarkers from the combined 141 sample set. As before, only those protein candidates that were nominated by all four methods were retained in the study. Because of the increase in statistical power afforded by the larger sample number, several hundred candidate proteins were found at the p<0.005 cutoff used previously. Therefore the nominal p-value cutoff was lowered to 0.001, and a set of 90 proteins differentiating Parkinson's disease from NC was found.

This set of 90 proteins for biological pathway enrichment was evaluated using the DAVID® BIOINFORMATICS RESOURCE®, version 6.7 (Huang, et al., 2008, Nat. Protocols 4, 44-57; Huang et al., 2009, Nucleic Acids Research 37, 1-13), and functional annotation tool, performed on the PANTHER® database (Muruganujan, et al., 2013, Nucleic Acids Research 41, D377-D386). In addition, this set of 90 proteins for enrichment in specific tissue and cell types was evaluated through DAVID® functional annotation tool, using UNIPROT® tissue designations. In both analyses, the 90 significant proteins were inputted as ‘gene list’ while all 968 proteins quantified in the study were inputted as ‘background gene list’, for purposes of assigning probabilities to the distribution of proteins observed in candidate Parkinson's disease biomarker list versus those expected under a random draw of 90 proteins from the 968 protein set.

### Age at Onset Analysis: Within Parkinson's Disease Patients

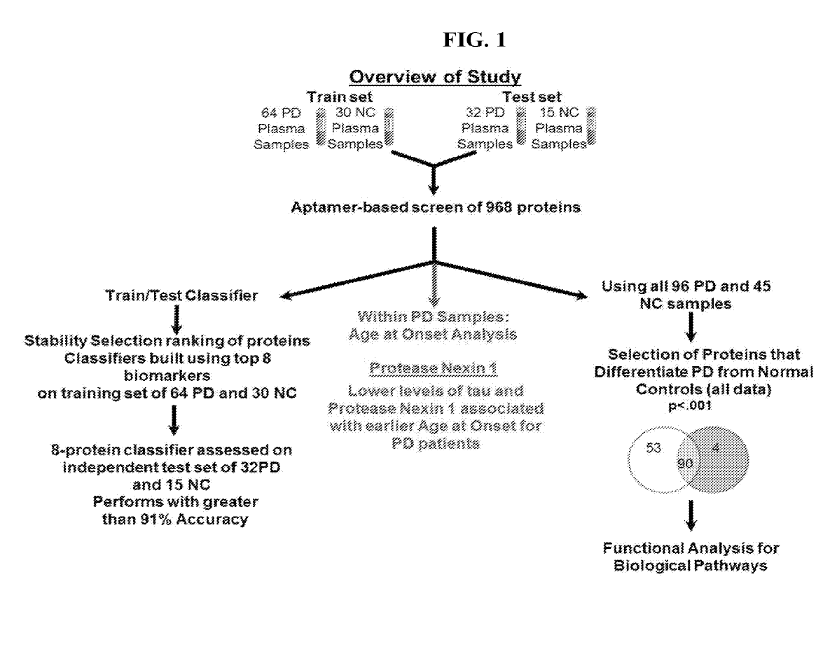

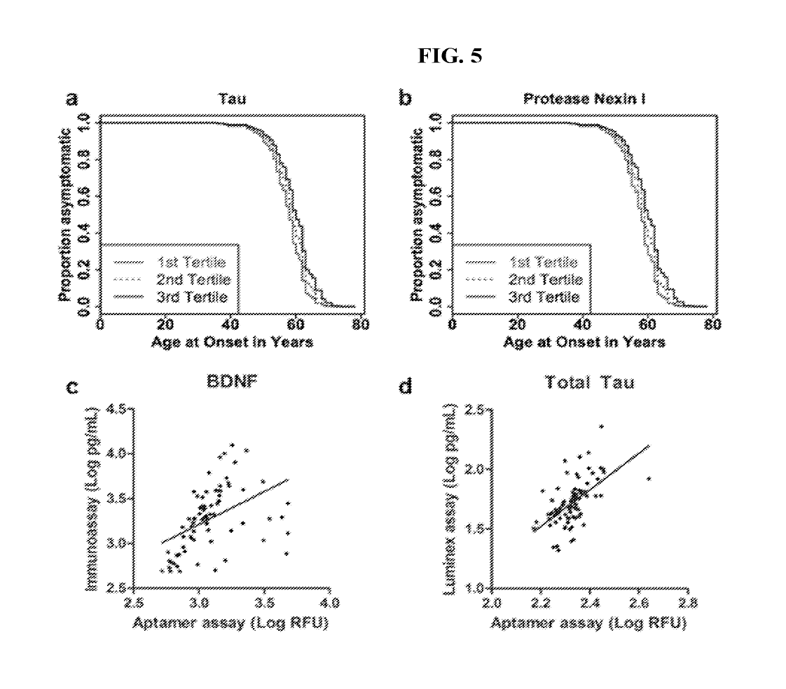

Age at Parkinson's disease onset was determined by patient report to the clinical research team. To evaluate whether any proteins in the 90 candidate biomarker list associated with age at Parkinson's disease onset, linear regression models associating age were used at Parkinson's disease onset with each of the candidate biomarkers, adjusting for age at plasma collection and sex (FIG. 1, middle panel). Proteins that were significant at the Bonferroni multiple testing correction level (p<0.005) are reported. In addition, Cox proportional hazards analyses were used to verify the association of age at Parkinson's disease onset with protein analytes nominated by linear regression. Cox proportional hazards models were also adjusted for age at plasma collection and sex and compared tertiles of protein measures. Cox regression survival curves were drawn using the R package “survival” (FIG. 5, Panels A and B) (Thermeau, A Package for Survival Analysis in S. R package version 2.37-7, http://CRAN.R-project.org/package=survival). All analyses were performed in R, and R-scripts are available upon request.

### Immunoassay Quantification of BDNF and Tau

BDNF blood plasma levels were measured using the human BDNF QUANTIKINE® ELISA Kit (R&D Systems—Minneapolis, Minn., USA) according to manufacturer's instructions. Samples were run in duplicate and a quality control (QC) cutoff of the coefficient of variation <0.2 were included in the final analysis. 108/116 (93%) of samples met this QC measure. Although this BDNF ELISA shows excellent relative quantitation, day-to-day variation makes the assay less robust for absolute quantitation. Therefore, all samples were processed on one lot of ELISAs, on the same day, by the same operator.

CSF total tau (t-tau) measures were obtained using the multiplex LUMINEX® XMAP® platform (Luminex Corp) using research only Fujirebio-Innogenetics INNO-BIA® AlZBIO3® immunoassay kit-based reagents as previously described (Kang, et al., 2013, JAMA Neurology, 70, 1277-1287; Shaw, et al, 2009, Annals of Neurology, 65, 403-413; Shaw, et al., 2011, Acta Neuropathol, 121, 597-609). Innogenetics kit reagents include XMAP® color-coded carboxylated microspheres, with each bead coupled with a monoclonal antibody for t-tau (AT120) and a corresponding vial with analyte specific biotinylated detector monoclonal antibody (HT7). All 80 CSF samples, calibrators, quality controls samples (75 μl of each) was analyzed in duplicate in each run as previously described (Shaw, et al., 2011, Acta Neuropathol. 121, 597-609).

### Results

### 968 Out of 1129 Protein Assays Demonstrate High Reproducibility on Aptamer-Based Screening Platform

To evaluate the reproducibility of the individual protein assays contained within this platform, three sets of triplicate identical aliquots were screened for a total of nine samples assayed for quality control (QC) purposes. Each set of triplicate aliquots came from one plasma draw from one subject, and the aptamer-based platform measures of these QC samples were separated in time so that both temporal and reagent batch variability were captured for the screening platform.

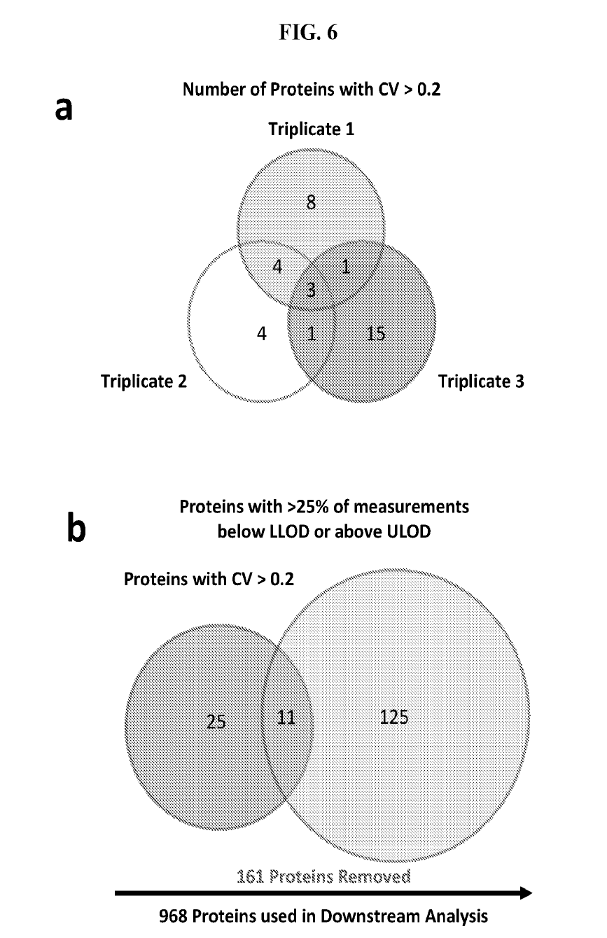

As shown in FIG. 6, Panel A, only 3/1129 (0.3%) of the protein assays on the screening platform demonstrated a coefficient of variation (CV) greater than 20% across all three triplicate sets. In addition, only 36/1129 (3.2%) of the protein assays showed a CV>20% on any one of the triplicate sets (FIG. 6, Panel B).

Having established the reproducibility of the individual protein assays within the aptamer-based platform, these assays were next examined to adequately cover the range of values found empirically in plasma samples, since assays may fail to robustly quantify their target proteins if the amount of protein in the biofluid of interest is at the limits of detection for the assay in question. Limits of detection were determined for each protein on the aptamer-based platform as previously described (Somalogic Inc. (2013) SOMASCAN® Technical White Paper. Retrieved from: http://www.somalogic.com/somalogic/media/Assets/PDFs/White-paper-2-22-13final.pdf). Comparing these limits of detection to the empirical measures obtained for samples, it was found that for 136/1129 (12.0%) of the protein assays, a significant proportion of samples (>25%) yielded measures outside the reliable limits of detection.

Because the over-riding goal was to keep only the most reliable protein assays in screen, both the 36 protein assays found to have high CV's in the QC samples and the 136 protein assays with many datapoints outside the limits of detection were removed from downstream consideration. These two filtering steps overlapped for 11 protein assays. Thus, a total of 161 protein assays on the aptamer-based screening platform were eliminated from consideration, leaving 968/1129 (85.7%) of the assays shown to perform with high reproducibility for downstream analyses (Table 3).

94 Candidate Plasma Proteins Differentiate Parkinson's Disease from Normal Controls in a Training Cohort

The cohort used in this study was carefully selected to reduce the possibility of bias and technical noise. All Parkinson's disease patients met the diagnostic criteria of the United Kingdom Parkinson's Disease Brain Bank. Moreover, all Parkinson's disease patients were part of a longitudinal, extensively-characterized cohort at the University of Pennsylvania, thus increasing the probability that Parkinson's disease is the true diagnosis in these subjects. Controls were recruited primarily from the unaffected spouses of Parkinson's disease cases from the same clinic, to control for environmental biases, and age and sex were matched between Parkinson's disease and control groups. Plasma samples from both groups were collected, processed, and stored in parallel, and all samples were assayed together, with operators blinded to disease status.

In a training cohort consisting of 64 Parkinson's disease and 30 NC subjects (Table 1), the concentrations of these 968 proteins were quantified using the aptamer-based assay (FIG. 1, left panel). To identify proteins that had significantly different concentrations in Parkinson's disease versus NC samples, Mann-Whitney U-tests (non-parametric), Student's t-tests (parametric), and permutation tests (10,000 resamples) were performed. 172 proteins were found to differentiate Parkinson's disease versus NC by all three statistical tests (nominal p<0.005).

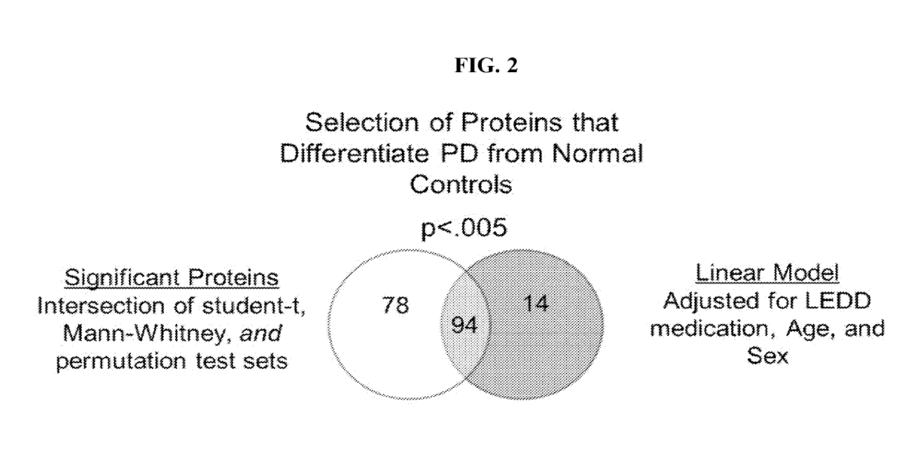

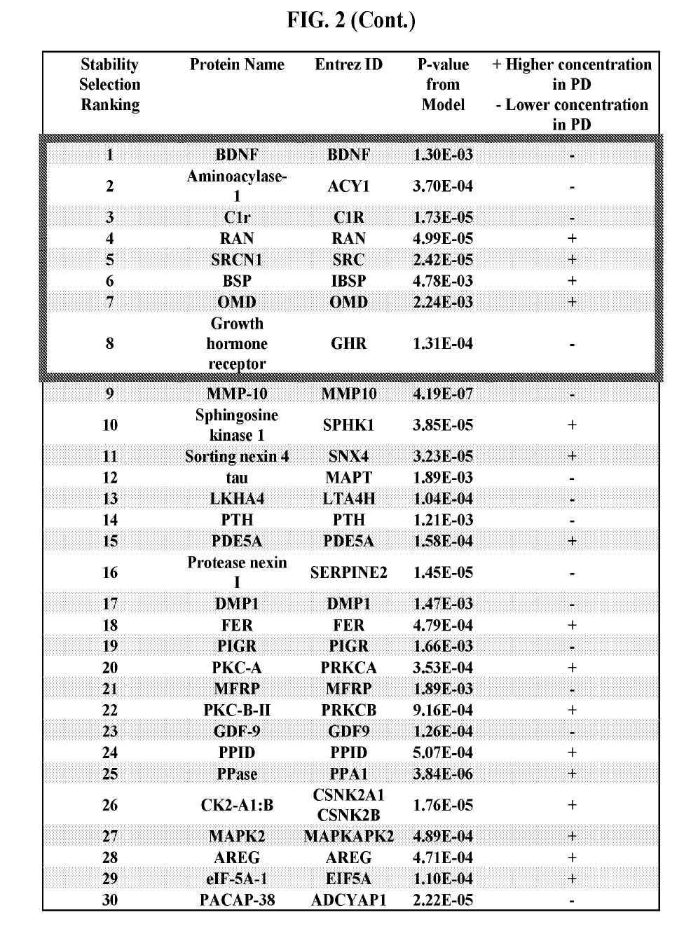

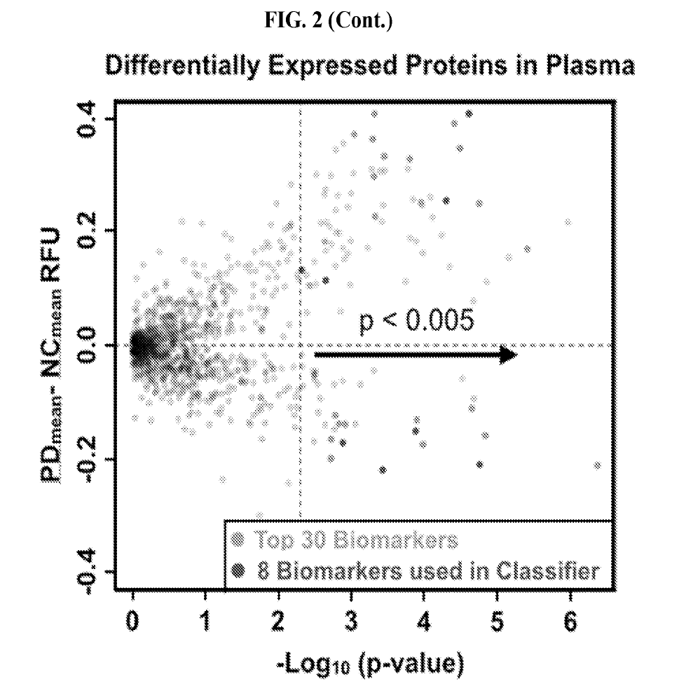

Because Parkinson's disease patients may take levodopa, a dopaminergic drug used to alleviate symptoms, this possible confounding effect on plasma protein expression, as well as demographic factors, was adjusted. Specifically, a linear model associating protein concentration with disease state was used to screen all 968 proteins, while also co-varying for the levodopa equivalent daily dose (LEDD) (Tomlinson, et al., 2010, Mov. Disord., 25, 2649-2653), age at plasma collection, and sex. It was found 108 proteins with differential expression in Parkinson's disease versus NC after adjusting for these covariates (nominal p<0.005). 94 of these proteins intersected with the previous 172 found using the three statistical tests (FIG. 2, Panel A). These 94 proteins represent the candidate plasma biomarkers differentiating Parkinson's disease from NC. The top 30 candidate biomarkers are listed in FIG. 2, Panel B.

Proteins that had higher plasma concentrations (n=69) in Parkinson's disease versus NC samples were disproportionately represented in the 94 candidate biomarkers compared to those that had lower concentrations (n=25). In contrast, among all 968 proteins quantified, the proportions with higher (n=459) and lower (n=509) mean concentrations in Parkinson's disease compared to NC were similar, suggesting that the enrichment in proteins with higher expression in Parkinson's disease is not the result of sample handling artifact (FIG. 2, Panel C).

### Stability Selection on Candidate Biomarker Proteins Yields an Eight-Protein Classifier

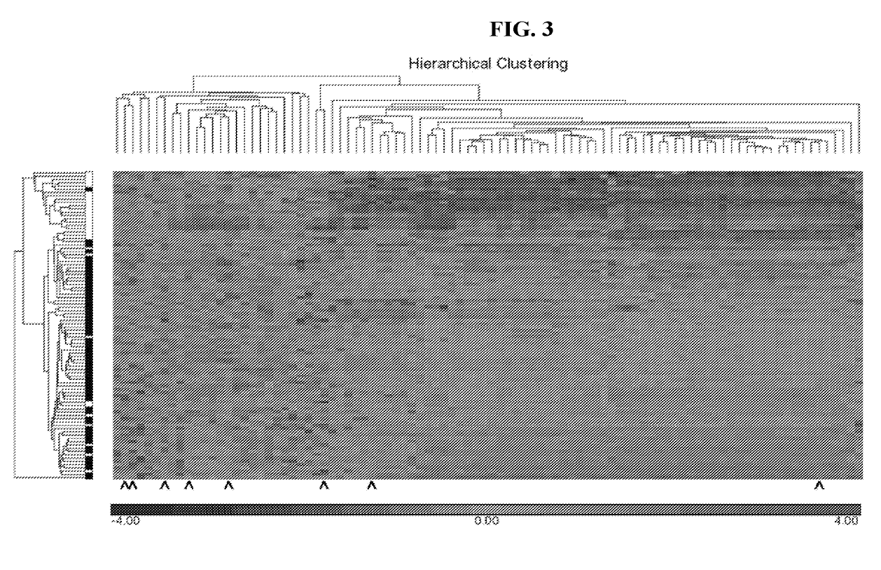

Hierarchical clustering on the 94 candidate biomarker proteins was performed to evaluate the correlation structure and associations between groups of proteins and disease state. As shown in FIG. 3, unsupervised clustering of training cohort samples using these 94 candidate proteins segregated Parkinson's disease patients (black) from NC (white), corroborating the utility of these 94 proteins in discriminating Parkinson's disease from NC. In addition, as expected, co-linearity among the 94 proteins was observed, indicating that there are redundancies and possible shared relationships among many of the candidate biomarkers (FIG. 3). In shown in FIG. 3, raw values for the 94 proteins differentiating Parkinson's disease from NC within the training cohort (n=64 Parkinson's disease, n=30 NC) were log-transformed, and these values were standardized by setting each protein to a mean of zero and standard deviation of 1. Both individual subjects and proteins were then hierarchically clustered by Euclidean distance using the average linkage. On the heatmap, blue represents lower expression levels, red represents higher expression levels, and grey represents intermediate levels. Each row represents one subject in the training cohort, and subjects are clustered on the Y-axis. Parkinson's disease patients are indicated by black blocks, while NC are indicated by white blocks. The separation of Parkinson's disease from NC corroborates the ability of the 94 candidate protein markers to distinguish Parkinson's disease from NC. Each column represents one protein in the set of 94 differentiating plasma proteins, and proteins are clustered on the X-axis. The top eight proteins identified using LASSO and Stability Selection for inclusion in the Parkinson's disease classifier are shaded in grey. These proteins are distributed across many of the clusters, suggesting that they comprise a sparse group of proteins that represent underlying differentiating signatures.

The observed correlations among 94 candidate biomarkers suggested that this candidate list could be reduced down to a smaller set of proteins for use in a diagnostic panel. From a practical standpoint, a smaller protein panel is easier and less expensive to implement in clinical settings. From an analytical perspective, a robust, stable, and “sparse” protein panel reduces the chance of over-fitting and removes redundant or noisy signals, thus improving the performance of a diagnostic classifier. To find this “sparse” panel, stability selection was used, a meta-statistical tool that identifies consistently important features by repeated sub-sampling of the data (FIG. 4, Panel A). As shown in FIG. 4, Panel A, Stability Selection, a meta-statistical tool that identifies consistently important features by repeated sub-sampling of the data, was used to rank the 94 candidate diagnostic biomarkers. Because cluster analyses showed high co-linearity among the 94 proteins, the LASSO method of feature selection was used to find a sparse panel of proteins for classifier training. In order to rank the 94 candidate biomarkers, 100,000 iterations of Stability Selection using LASSO were run. At each iteration, 10% of the samples and 30% of the proteins were randomly removed, and the LASSO regularization method was used to identify a sparse set of proteins using the remaining jack-knifed data. Proteins were then ranked by the number of times LASSO included the protein in the model across the 100,000 iterations.

To rank the 94 candidate biomarkers within stability selection, the LASSO (Least Absolute Shrinkage and Selection Operator) technique was used fit to jack-knifed data across 100,000 iterations (Tibshirani, et al., 1996, Journal of the Royal Statistical Society: Series B (Statistical Methodology), 58, 267-288).

The optimal number of biomarkers to be used in the diagnostic panel were then determined by creating classifiers using n=1 to n=30 of the top-ranked biomarkers. Both Support Vector Machine (SVM) and Logistic Regression classifiers were evaluated (FIG. 4, Panel E), and a 10-fold cross-validation method was used on the training dataset to assess the performance of classifiers (FIG. 4, Panel B). As shown in FIG. 4, Panel B, Support Vector Machine (SVM) classifiers using the Radial Based Kernel were built from training set data (n=64 Parkinson's disease, n=30 NC) using n=1 to n=30 of the top ranked biomarkers along with age and sex as covariates. 10-fold cross-validation was used to assess performance. The Area under the Curve (AUC, black trace) and Accuracy (brown trace) is plotted for of each classifier using various numbers of protein features. An eight-protein classifier achieved the highest performance as measured by AUC (0.98) and Accuracy (94%).

### The Average Performance for the Classifiers was Calculated Across all Iterations.

Using SVM classifiers in training cohort dataset, it was found that a panel of just eight top plasma biomarkers gave us the best average performance measured by area under the curve (AUC, 0.983±0.027) and classification accuracy (0.935±0.056) (FIG. 4, Panel C). As shown in FIG. 4, Panel C, a Logistic Regression classifier (red curve) was also built on training set data (n=64 Parkinson's disease, n=30 NC), using the top 8 proteins, with age and sex as covariates. Performance for both classifiers was assessed using 10-fold cross-validation within the training set. The ROC curves for the SVM (black curve) and Logistic Regression classifiers are shown. The SVM classifier achieved an AUC=0.98, Accuracy=94%, Sensitivity=97% and Specificity=87%. The Logistic Regression classifier achieved an AUC=0.96, Accuracy=90%, Sensitivity=91%, and Specificity=90%. Specifically, plasma measures of brain-derived neurotrophic factor (BDNF), aminoacylase-1, complement component 1 r subcomponent (Cir), ras-related nuclear protein (RAN), SRC kinase signaling inhibitor 1 (SRCN1), bone sialoprotein (BSP), osteomodulin (OMD), and growth hormone receptor, in aggregate, classified Parkinson's disease versus NC samples with high accuracy.

Comparing different classifiers, SVM exhibited slightly stronger performance than Logistic Regression classifiers (FIG. 4, Panel C) in the training set, although both types of classifiers demonstrated >90% accuracy in 10-fold cross-validation.

### Applying an 8-Protein Classifier to an Independent Test Set of 47 Samples Confirms High Classification Accuracy

Due to its high classification accuracy, an eight-plasma protein panel in training dataset was next evaluated with an independent test set of 32 Parkinson's disease samples versus 15 NC samples (Table 1)(FIG. 1, left panel). These samples were collected from the same clinical sites during the same time period, minimizing noise from heterogeneity in sample collection, but they were never used in the analysis stream used to build the classifier and represent a separate replication set.

As shown in FIG. 4, Panel D, the exact same eight-protein SVM and Logistic Regression classifiers developed in the training set were then used to predict disease state in the independent test set of 32 Parkinson's disease and 15 NC subjects. The ROC performance for the SVM and Logistic Regression classifiers in this test set are plotted in black and red, respectively. The SVM classifier achieved an AUC=0.90, Accuracy=91%, Sensitivity=97% and Specificity=80%.

The Logistic Regression classifier achieved an AUC=0.88, Accuracy=87%, Sensitivity=91% and Specificity=80%. Classification accuracy remained high in this independent test set. Specifically, for the SVM classifier, an accuracy of 91% was obtained (sensitivity 0.97, specificity 0.80), with an AUC of 0.90 in this second set of samples. The Logistic Regression classifier also demonstrated strong performance in the test set, with an accuracy of 87% (sensitivity 0.91, specificity 0.80) and an AUC of 0.88.

Pathway Analysis Performed on Biomarkers from the Combined Dataset Implicates Parkinson's Disease and Growth Factor Signaling Pathways

Plasma-based biomarkers may be useful as clinical tools, functioning in a biologically-agnostic manner. However, unbiased screening methods for their discovery may also lead to insights into disease pathogenesis and potential therapeutic targets. To explore this aspect of the data, further analyses were performed using plasma proteins differentially expressed in Parkinson's disease versus NC. To make the best use of sample data for these exploratory purposes, the training and test sets were combined for a total of 96 Parkinson's disease and 45 NC samples (Table 1)(FIG. 1, right panel). The Mann-Whitney U-tests, Student's t-tests, and permutation tests were re-run on this 141-sample set. 143 proteins were found to differentiate Parkinson's disease versus NC on this combined dataset by all three statistical tests (p<0.001). As before with the training set, this 141-sample combined set was evaluated in the linear model associating protein concentration with disease state while co-varying for LEDD, age at plasma collection, and sex. 94 proteins differentiated Parkinson's disease versus NC in this linear model, with 90 of these proteins intersecting with the 143 proteins found using the three statistical tests (FIG. 1, right panel). These 90 top plasma proteins differentiating Parkinson's disease from NC were used in downstream biological pathway analyses (Table 4).

Strikingly, one of the top pathways identified in the PANTHER database (29) to be enriched in this set of 90 proteins was Parkinson's disease itself, with a 2.7 fold-enrichment of Parkinson's disease-affiliated genes (Table 5) in candidate biomarker list (p=0.008). Additionally, pathway analyses found enrichment of multiple trophic factor signaling pathways (e.g. Platelet-derived growth factor (PDGF) signaling pathway p=0.028; Vascular endothelial growth factor (VEGF) signaling pathway p=0.040; Epidermal growth factor (EGF) receptor signaling pathway p=0.050) within the top candidate biomarker list. Finally, the highest tissue enrichment seen in set of 90 proteins was for brain-associated tissues (fetal brain cortex p=3.56×10⁻¹²; Cajal-Retzius cell p=1.30×10⁻⁹; brain p=4.99×10⁻⁵). Pathways and tissues found to be significantly enriched in the 90 top proteins differentiating Parkinson's disease versus NC are summarized in Table 2.

Unbiased pathway analysis of the top 90 proteins found here to differentiate Parkinson's disease and NC plasma samples led to the striking finding that these proteins were highly enriched in lists of genes/proteins previously implicated in Parkinson's disease itself. Such a finding adds confidence to the strategy for the discovery of candidate Parkinson's disease markers. In addition, it suggests that both the pathway analysis algorithms and the databases on which they are based can lead to true biological insight. Intriguingly, pathway analysis of the top plasma biomarkers also demonstrated enrichment in multiple trophic factor pathways.

Plasma Levels of Tau and Protease Nexin 1 Associate with Age at Onset in Parkinson's Disease

Dopaminergic neuron loss likely begins well before the onset of clinical Parkinson's disease (Cheng, et al., 2010, Ann Neurol., 67, 715-725; Fearnley, et al., 1991, Brain, 114, 2283-2301). As a consequence, one might expect that some of the proteins differentiating Parkinson's disease versus NC might also show correlations with the age at Parkinson's disease onset, since the age at clinical onset basically represents the moment when enough dopaminergic neurons have been lost to make disease manifest. Top 90 plasma proteins differentiating Parkinson's disease versus NC were tested for ability to predict the age at Parkinson's disease onset in a linear model co-varying for age at plasma sampling, sex, and LEDD, using the combined dataset.

As shown in FIG. 5, Panels A and B, lower concentrations of protease nexin 1 and tau were found to associate with an earlier age at Parkinson's disease onset in both a linear regression model (p=0.0041 for protease nexin 1, p=0.0039 for tau) and in a Cox proportional hazards model (p=0.052 and hazards ratio=0.7649 for each tertile increase in protease nexin 1, p=0.042 and hazards ratio=0.7750 for each tertile increase in tau). Protease nexin 1 and tau were also previously found to be lower in concentration in Parkinson's disease samples compared to NC, with both proteins within the top 30 Stability-Selection-ranked proteins differentiating Parkinson's disease from NC (FIG. 2, Panel C).

Tau and protease nexin 1 were found to correlate with age at Parkinson's disease onset, with lower levels found in Parkinson's disease compared with NC, and lower levels found in Parkinson's disease subjects with an earlier age at disease onset. This finding increases the confidence in these two proteins since the gradation of levels within Parkinson's disease according to one measure of pathophysiological severity (earlier age at onset) suggests that the differences between Parkinson's disease and NC are not due to a hidden confounding variable differentiating these two groups. Moreover, it is likely that neurodegeneration begins years before the clinical diagnosis of Parkinson's disease, with an estimated 50% of dopaminergic neurons lost before the onset of motor symptoms. This situation offers the possibility of a window of therapeutic opportunity before the onset of symptoms, if pre-symptomatic diagnosis or risk assessment could be achieved. The fact that these two markers associate significantly with age at disease onset suggests that they may be markers of disease risk as well as disease state.

### Immunoassays for BDNF and Tau Corroborate Aptamer-Based Measures

Key plasma proteins differentiating Parkinson's disease from NC through unbiased screening on the aptamer-based platform were retested using conventional immunoassays.

As shown in FIG. 5, Panel C, measures for top differentiating protein, BDNF, demonstrated moderately strong correlation comparing the aptamer-based assay with a conventional immunoassay. Specifically, between measures on the two assays, the Spearman correlation coefficient for BDNF was 0.62 (p<0.001).

Since no universally-accepted assays for plasma tau exist, CSF tau was used for comparison between a LUMINEX®-based immunoassay and for an aptamer-based immunoassay. As shown in FIG. 5, Panel D, using a set of 80 CSF samples (Table 6 for sample details) for which duplicate aliquots were measured with both assays, correlation was moderately strong, with a Spearman correlation coefficient of 0.60 and p-value<0.001.

BDNF and tau were chosen for second-method corroboration because of the extensive literature implicating these two proteins in neurodegeneration. As mentioned previously, in multiple animal models, augmentation of BDNF has been shown to protect against neurodegeneration. The microtubule-associated protein tau, encoded by the gene MAPT, has also been implicated in neurodegenerative disease processes for more than 20 years. In Parkinson's disease, genotypes and haplotypes at the MAPT locus have been associated with Parkinson's disease in multiple genomewide association studies. Moreover. CSF levels of both total tau and tau phosphorylated at threonine-181 are significantly decreased in Parkinson's disease. While no well-validated assay for serum or plasma tau currently exists, in pilot studies, serum tau has been reported to differ in subjects with brain injury compared to normal controls (Henriksen, et al., 2014, Alzheimer's & Dementia: The Journal of the Alzheimer's Association, 10, 115-131). Thus, current finding of decreased tau plasma levels in Parkinson's disease patients further supports a role for this protein in disease pathophysiology.

### Robustness of the Biomarker Panel

Several methodological points may account for the relative robustness of the biomarker panel. First, potential biomarkers for those proteins with the most robust performance were filtered on the screening platform. Second, the meta-statistical technique of stability selection was employed to select the panel of top biomarkers. This technique, which consists of iteratively sub-sampling subjects and proteins (thus “jack-knifing” the data), enriches for biomarkers robust to differences in the specific sample sets and other protein markers used. Third, compared to mRNAs, proteins are relatively stable and long-lived; as a consequence, protein measurements are the basis of most clinical lab tests in use today. It is also noted here that the biomarker panel is based on plasma proteins. As such, these biomarkers can be obtained easily and inexpensively by routine blood draw in most clinical contexts, offering a significant practical advantage over CSF or imaging-based measures.

### Reproducibility of Somalogic Aptamer-Based Assay

The aptamer-based assay used for the biomarker discovery is very new, and has been commercialized by Somalogic, Inc., in its present form only two years ago, after more than a decade of research and development. To evaluate its reliability and reproducibility, a series of quality-control (QC) experiments were conducted independently of Somalogic, to ensure the integrity of the findings that the aptamer-based assay is better than other methods to quantitate many proteins in parallel. The other methods including mass spectrometry-based proteomics and multiplexed immunoassays are notoriously difficult to reproduce.

First, in the initial aptamer-based assay run, three samples were assayed in triplicate (3×3=9 QC samples), masking the fact that these were replicate samples and separating the replicates across batches. Then coefficients of variation (CV) for the triplicate samples were calculated. Only 36/1129 (3.1%) proteins assayed demonstrated a CV >0.2. By way of comparison, when identical QC experiments using a widely-used commercially-available multiplex immunoassay (Rules-Based Medicine, Inc.) were performed, >30% of the proteins assayed failed this measure.

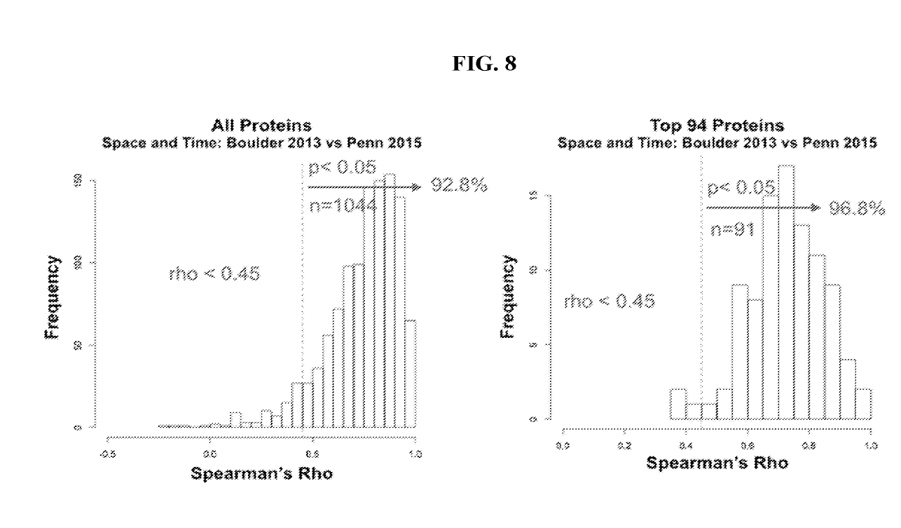

Second, the University of Pennsylvania (UPenn) has partnered with Somalogic to make the aptamer-based platform available locally (UPenn is the only site outside of Somalogic company headquarters in Boulder, Colo., with this capability). The reproducibility of this assay was evaluated across time (Boulder, Colo., in 2013, vs. Boulder, Colo., in 2015), across space (Boulder, Colo., in 2015, vs. UPenn in 2015), and across both time and space (Boulder, Colo., in 2013, vs. UPenn in 2015). Specifically, replicate aliquots of 20 samples included in the 2013 run were assayed using the aptamer-based assay, performed in Boulder and at UPenn. As summarized in FIG. 8, reproducibility across the entire assay was reasonably high (>90% of proteins with significant correlation across space and time), and across the top 94 proteins found to distinguish PD from normal controls, reproducibility was even higher (>95% of proteins with significantly correlated measurements across space and time). For the top 8 proteins of greatest interest, all showed good reproducibility.

TABLE 1

| | | | | | | Age at | | |
|---|---|---|---|---|---|---|---|---|
| | | | | Age at | | Disease | | UPDRS |
| | | | | plasma | | Onset | Disease | Motor |
| | | Number | | Mean | | Mean | Duration | Score |
| | | Female/ | | years ± | | years ± | Mean | Mean score ± |
| | | Male | P | SD | P | SD | years ± SD | SD |
| Train | PD | 64 | 1^{a} | 69.47 ± 7.57 | 0.96^{b} | 58.28 ± 7.87 | 11.19 ± 5.45 | 26.23 ± 13.38 |
| Set | | 27/37 | | | | | | |
| | NC | 30 | | 70.80 ± 10.81 | | N/A | N/A | N/A |
| | | 13/17 | | | | | | |
| Test | PD | 32 | 0.53^{a} | 70.78 ± 7.48 | 0.53^{b} | 59.75 ± 10.47 | 11.03 ± 6.34 | 25.38 ± 10.49 |
| Set | | 15/17 | | | | | | |
| | NC | 15 | | 68.87 ± 8.49 | | N/A | N/A | N/A |
| | | 9/6 | | | | | | |
| Total | PD | 96 | 0.59^{a} | 69.91 ± 7.52 | 0.65^{b} | 58.77 ± 8.79 | 11.14 ± 5.73 | 25.95 ± 12.44 |
| Data | | 42/54 | | | | | | |
| | NC | 45 | | 70.16 ± 10.04 | | N/A | N/A | N/A |
| | | 22/23 | | | | | | |

TABLE 2

| | Proteins | | |
|---|---|---|---|
| | (UNIPROT | Fold | |
| PANTHER Pathway | Accession) | Enrichment | P-Value |
| Angiotensin II-stimulated | P41743, P17252, | 4.74 | 3.54E−03 |
| signaling through G | P28482, P27361, | | |
| proteins and beta-arrestin | P05771, P25098 | | |
| Parkinson disease | P12931, P28482, | 2.73 | 7.71E−03 |
| | P41240, P06241, | | |
| | P27361, P31946, | | |
| | P68400, P25098, | | |
| | P07948 | | |
| B cell activation | P41743, P17252, | 2.63 | 1.79E−02 |
| | P28482, P27361, | | |
| | P05771, Q06187, | | |
| | P07948, P63000 | | |
| PDGF signaling pathway | P49137, P41743, | 2.43 | 2.80E−02 |
| | O15530, P17252, | | |
| | P28482, P27361, | | |
| | P05771, P49840 | | |
| VEGF signaling pathway | P49137, P41743, | 2.51 | 3.98E−02 |
| | P17252, P28482, | | |
| | P27361, P05771, | | |
| | Q9NYA1 | | |
| T cell activation | P41743, P17252, | 2.40 | 4.89E−02 |
| | P28482, P41240, | | |
| | P27361, P05771, | | |
| | P63000 | | |
| EGF receptor signaling | P41743, P15514, | 2.18 | 4.98E−02 |
| pathway | P17252, P28482, | | |
| | P27361, P31946, | | |
| | P05771, P63000 | | |

| Top 5 Enriched Tissues | | Fold | |
|---|---|---|---|
| (UNIPROT Tissue) | Number of Proteins | Enrichment | P-Value |
| Fetal brain cortex | 25 | 4.70 | 3.56E−12 |
| Cajal-Retzius cell | 21 | 4.45 | 1.30E−09 |
| Platelet | 23 | 3.27 | 1.40E−07 |
| Urinary bladder | 10 | 5.86 | 8.03E−06 |
| Brain | 55 | 1.51 | 4.99E−05 |

TABLE 3

| | | | >25% |
|---|---|---|---|
| | | | data |
| | | CV > | within |
| Protein Name | Entrez Name | 0.2 | LOD |
| C4b | C4A C4B | 1 | 1 |
| Coagulation Factor XI | F11 | 0 | 0 |
| CTACK | CCL27 | 0 | 0 |
| Endostatin | COL18A1 | 0 | 0 |
| TIMP-1 | TIMP1 | 0 | 0 |
| tPA | PLAT | 0 | 0 |
| EG-VEGF | PROK1 | 0 | 0 |
| TIMP-2 | TIMP2 | 0 | 0 |
| TGF-b1 | TGFB1 | 0 | 0 |
| VEGF sR3 | FLT4 | 0 | 0 |
| C5 | C5 | 0 | 0 |
| Apo E | APOE | 0 | 0 |
| BDNF | BDNF | 0 | 0 |
| bFGF-R | FGFR1 | 0 | 1 |
| C8 | C8A C8B C8G | 0 | 0 |
| Cathepsin G | CTSG | 0 | 1 |
| CXCL16 soluble | CXCL16 | 0 | 0 |
| FGF-10 | FGF10 | 0 | 0 |
| FGF-8B | FGF8 | 0 | 1 |
| GIIE | PLA2G2E | 0 | 0 |
| GV | PLA2G5 | 0 | 0 |
| IL-12 | IL12A IL12B | 0 | 1 |
| MIP-3a | CCL20 | 0 | 0 |
| SAP | APCS | 0 | 0 |
| SCF sR | KIT | 0 | 0 |
| TIMP-3 | TIMP3 | 0 | 0 |
| TWEAK | TNFSF12 | 0 | 0 |
| Angiopoietin-4 | ANGPT4 | 0 | 0 |
| Cadherin E | CDH1 | 0 | 0 |
| GFRa-3 | GFRA3 | 0 | 1 |
| Ephrin-B3 | EFNB3 | 0 | 0 |
| GFRa-2 | GFRA2 | 0 | 0 |
| 6Ckine | CCL21 | 0 | 0 |
| RANTES | CCL5 | 0 | 0 |
| HMG-1 | HMGB1 | 0 | 0 |
| OPG | TNFRSF11B | 0 | 0 |
| b-Endorphin | POMC | 0 | 0 |
| Factor I | CFI | 0 | 0 |
| IGFBP-2 | IGFBP2 | 0 | 0 |
| IGFBP-3 | IGFBP3 | 0 | 0 |
| Leptin | LEP | 0 | 0 |
| MCP-1 | CCL2 | 0 | 0 |
| MMP-9 | MMP9 | 0 | 0 |
| Myeloperoxidase | MPO | 0 | 0 |
| PRL | PRL | 0 | 0 |
| ROR1 | ROR1 | 0 | 0 |
| VEGF | VEGFA | 0 | 0 |
| 4-1BB | TNFRSF9 | 0 | 0 |
| 4-1BB ligand | TNFSF9 | 0 | 0 |
| Angiopoietin-2 | ANGPT2 | 0 | 0 |
| B7 | CD80 | 0 | 1 |
| CD30 | TNFRSF8 | 0 | 1 |
| CLF-1/CLC Complex | CRLF1 CLCF1 | 0 | 0 |
| Cystatin C | CST3 | 0 | 0 |
| Dtk | TYRO3 | 0 | 0 |
| eIF-5 | EIF5 | 0 | 0 |
| Ephrin-A4 | EFNA4 | 0 | 0 |
| Ephrin-A5 | EFNA5 | 0 | 0 |
| ERBB2 | ERBB2 | 0 | 0 |
| ERBB3 | ERBB3 | 0 | 0 |
| ERBB4 | ERBB4 | 0 | 0 |
| GA733-1 protein | TACSTD2 | 0 | 0 |
| gp130 soluble | IL6ST | 0 | 0 |
| HO-2 | HMOX2 | 0 | 0 |
| HPV E7 Type 16 | Human-virus | 0 | 0 |
| HPV E7 Type18 | Human-virus | 0 | 1 |
| HSP 90a/b | HSP90AA1 HSP90AB1 | 0 | 0 |
| IL-1 R AcP | IL1RAP | 0 | 0 |
| IL-10 Rb | IL10RB | 0 | 1 |
| IL-12 Rb1 | IL12RB1 | 0 | 0 |
| IL-13 Ra1 | IL13RA1 | 0 | 0 |
| IL-2 sRg | IL2RG | 0 | 0 |
| Layilin | LAYN | 0 | 0 |
| Lymphotoxin b R | LTBR | 0 | 0 |
| Macrophage mannose | MRC1 | 0 | 0 |
| receptor | | | |
| M-CSF R | CSF1R | 0 | 1 |
| MSP R | MST1R | 0 | 1 |
| PAFAH beta subunit | PAFAH1B2 | 0 | 0 |
| P-Cadherin | CDH3 | 0 | 1 |
| PKC-A | PRKCA | 0 | 0 |
| PKC-Z | PRKCZ | 0 | 0 |
| Rab GDP dissociation | GDI2 | 0 | 0 |
| inhibitor beta | | | |
| sICAM-3 | ICAM3 | 0 | 1 |
| suPAR | PLAUR | 0 | 0 |
| Tau | MAPT | 0 | 0 |
| TNF sR-I | TNFRSF1A | 0 | 0 |
| TrkC | NTRK3 | 0 | 0 |
| BCMA | TNFRSF17 | 0 | 0 |
| Bone proteoglycan II | DCN | 0 | 0 |
| Calpain I | CAPN1 CAPNS1 | 0 | 0 |
| CK-MM | CKM | 0 | 0 |
| Cripto | TDGF1 | 0 | 0 |
| ERBB1 | EGFR | 0 | 0 |
| HGF | HGF | 0 | 0 |
| HSP 60 | HSPD1 | 0 | 0 |
| iC3b | C3 | 1 | 0 |
| IGFBP-5 | IGFBP5 | 0 | 0 |
| IGFBP-6 | IGFBP6 | 0 | 0 |
| MIA | MIA | 0 | 0 |
| NEUREGULIN-1 | NRG1 | 1 | 0 |
| NPS-PLA2 | PLA2G2A | 0 | 0 |
| OSM | OSM | 0 | 1 |
| PECAM-1 | PECAM1 | 0 | 1 |
| Persephin | PSPN | 0 | 0 |
| PF-4 | PF4 | 0 | 0 |
| Protein S | PROS1 | 0 | 1 |
| TACI | TNFRSF13B | 0 | 1 |
| TECK | CCL25 | 0 | 0 |
| Thyroxine-Binding | SERPINA7 | 0 | 0 |
| Globulin | | | |
| TNFSF18 | TNFSF18 | 0 | 0 |
| CNTFR alpha | CNTFR | 0 | 0 |
| EMAP-2 | AIMP1 | 0 | 0 |
| EPO-R | EPOR | 0 | 0 |
| G-CSF-R | CSF3R | 0 | 0 |
| IL-1F7 | IL1F7 | 0 | 0 |
| Laminin | LAMA1 LAMB1 LAMC1 | 0 | 0 |
| MICA | MICA | 0 | 0 |
| NADPH-P450 | POR | 0 | 0 |
| Oxidoreductase | | | |
| NANOG | NANOG | 0 | 0 |
| NKp44 | NCR2 | 1 | 0 |
| Noggin | NOG | 0 | 1 |
| NovH | NOV | 0 | 0 |
| Siglec-6 | SIGLEC6 | 0 | 0 |
| Siglec-7 | SIGLEC7 | 0 | 0 |
| Sonic Hedgehog | SHH | 0 | 0 |
| TSLP R | CRLF2 | 0 | 0 |
| ULBP-3 | ULBP3 | 0 | 0 |
| Activin A | INHBA | 0 | 0 |
| Apo A-I | APOA1 | 0 | 0 |
| Azurocidin | AZU1 | 0 | 0 |
| BMP-14 | GDF5 | 1 | 1 |
| C1q | C1QA C1QB C1QC | 0 | 1 |
| C3 | C3 | 1 | 0 |
| C3adesArg | C3 | 0 | 1 |
| DRR1 | FAM107A | 0 | 0 |
| FGF-18 | FGF18 | 0 | 0 |
| FGF-19 | FGF19 | 0 | 0 |
| FGF-20 | FGF20 | 0 | 0 |
| FGF9 | FGF9 | 0 | 1 |
| GDF-11 | GDF11 | 0 | 0 |
| Hemopexin | HPX | 0 | 0 |
| HIV-2 Rev | Human-virus | 0 | 0 |
| I-309 | CCL1 | 0 | 0 |
| IGFBP-1 | IGFBP1 | 0 | 0 |
| IL-10 | IL10 | 0 | 0 |
| IL-16 | IL16 | 0 | 0 |
| IL-17F | IL17F | 0 | 0 |
| IL-22 | IL22 | 0 | 0 |
| Lactoferrin | LTF | 0 | 0 |
| LAG-1 | CCL4L1 | 0 | 0 |
| LD78-beta | CCL3L1 | 0 | 0 |
| MCP-2 | CCL8 | 0 | 1 |
| MMP-3 | MMP3 | 0 | 0 |
| MMP-7 | MMP7 | 0 | 0 |
| NAP-2 | PPBP | 0 | 0 |
| SOD | SOD1 | 0 | 0 |
| Alkaline phosphatase bone | ALPL | 0 | 0 |
| Fibrinogen | FGA FGB FGG | 0 | 0 |
| Apo B | APOB | 0 | 0 |
| ACE2 | ACE2 | 0 | 0 |
| Activin RIB | ACVR1B | 0 | 1 |
| ADAMTS-4 | ADAMTS4 | 0 | 0 |
| Angiopoietin-1 | ANGPT1 | 0 | 0 |
| ART | AGRP | 0 | 0 |
| BCAM | BCAM | 0 | 0 |
| Cadherin-5 | CDH5 | 0 | 0 |
| CD97 | CD97 | 0 | 0 |
| COMMD7 | COMMD7 | 0 | 0 |
| EDA | EDA | 0 | 0 |
| Fractalkine/CX3CL-1 | CX3CL1 | 0 | 0 |
| HAI-1 | SPINT1 | 0 | 0 |
| IL-27 | IL27 EBI3 | 0 | 0 |
| Kallikrein 11 | KLK11 | 0 | 0 |
| Kallikrein 4 | KLK4 | 0 | 0 |
| kallikrein 8 | KLK8 | 0 | 0 |
| Ku70 | XRCC6 | 0 | 0 |
| Lipocalin 2 | LCN2 | 0 | 0 |
| Met | MET | 0 | 0 |
| MMP-17 | MMP17 | 0 | 1 |
| OX40 Ligand | TNFSF4 | 0 | 1 |
| sFRP-3 | FRZB | 0 | 0 |
| sICAM-2 | ICAM2 | 0 | 0 |
| SPINT2 | SPINT2 | 0 | 0 |
| sTie-1 | TIE1 | 0 | 0 |
| Ubiquitin+1 | RPS27A | 0 | 0 |
| WIF-1 | WIF1 | 0 | 0 |
| AIF1 | AIF1 | 0 | 0 |
| ARGI1 | ARG1 | 0 | 0 |
| C5a | C5 | 0 | 0 |
| CHK1 | CHEK1 | 0 | 1 |
| ERK-1 | MAPK3 | 0 | 0 |
| Glucocorticoid receptor | NR3C1 | 0 | 0 |
| Hat1 | HAT1 | 0 | 0 |
| HDAC8 | HDAC8 | 0 | 0 |
| Karyopherin-a2 | KPNA2 | 0 | 0 |
| MEK1 | MAP2K1 | 1 | 0 |
| MOZ | KAT6A | 0 | 1 |
| PKC-D | PRKCD | 0 | 1 |
| RAC1 | RAC1 | 0 | 0 |
| RAD51 | RAD51 | 0 | 0 |
| TBP | TBP | 0 | 0 |
| Topoisomerase I | TOP1 | 0 | 0 |
| UBC9 | UBE2I | 0 | 0 |
| YES | YES1 | 0 | 0 |
| a1-Antichymotrypsin | SERPINA3 | 0 | 1 |
| C7 | C7 | 0 | 0 |
| Cardiotrophin-1 | CTF1 | 0 | 1 |
| CCL28 | CCL28 | 0 | 0 |
| CD22 | CD22 | 0 | 0 |
| HCC-1 | CCL14 | 0 | 0 |
| IL-4 | IL4 | 0 | 0 |
| Midkine | MDK | 0 | 0 |
| MPIF-1 | CCL23 | 0 | 0 |
| PCNA | PCNA | 0 | 0 |
| sRANKL | TNFSF11 | 0 | 0 |
| PAI-1 | SERPINE1 | 0 | 0 |
| Apo E3 | APOE | 0 | 0 |
| Apo E4 | APOE | 0 | 0 |
| Artemin | ARTN | 0 | 0 |
| Cytochrome c | CYCS | 0 | 0 |
| Cytochrome P450 3A4 | CYP3A4 | 0 | 0 |
| DAN | NBL1 | 0 | 0 |
| ER | ESR1 | 0 | 0 |
| Factor D | CFD | 0 | 1 |
| Growth hormone receptor | GHR | 0 | 0 |
| GX | PLA2G10 | 0 | 1 |
| IGFBP-4 | IGFBP4 | 0 | 0 |
| IGF-I | IGF1 | 0 | 0 |
| Luteinizing hormone | CGA LHB | 0 | 0 |
| MMP-8 | MMP8 | 0 | 0 |
| NG36 | EHMT2 | 0 | 0 |
| Properdin | CFP | 0 | 1 |
| Protein C | PROC | 0 | 0 |
| PTHrP | PTHLH | 0 | 0 |
| SCGF-beta | CLEC11A | 0 | 0 |
| VCAM-1 | VCAM1 | 0 | 0 |
| TNFSF15 | TNFSF15 | 0 | 0 |
| ALK-1 | ACVRL1 | 0 | 0 |
| AREG | AREG | 0 | 0 |
| BMP-7 | BMP7 | 0 | 0 |
| CD36 ANTIGEN | CD36 | 0 | 0 |
| contactin-1 | CNTN1 | 0 | 0 |
| CTGF | CTGF | 0 | 0 |
| Desmoglein-1 | DSG1 | 0 | 1 |
| EDAR | EDAR | 0 | 0 |
| ENA-78 | CXCL5 | 0 | 1 |
| ESAM | ESAM | 0 | 0 |
| Galectin-4 | LGALS4 | 0 | 0 |
| Gro-a | CXCL1 | 0 | 0 |
| Gro-b/g | CXCL3 CXCL2 | 0 | 0 |
| Histone H1.2 | HIST1H1C | 0 | 0 |
| ICOS | ICOS | 0 | 0 |
| IFN-g | IFNG | 0 | 0 |
| IL-1 sRI | IL1R1 | 0 | 0 |
| IL-17 sR | IL17RA | 0 | 0 |
| IL-18 Rb | IL18RAP | 0 | 1 |
| IL-1Rrp2 | IL1RL2 | 0 | 0 |
| JAM-B | JAM2 | 0 | 0 |
| JAM-C | JAM3 | 0 | 0 |
| LSAMP | LSAMP | 0 | 0 |
| MBL | MBL2 | 0 | 0 |
| NKp30 | NCR3 | 0 | 0 |
| PD-L2 | PDCD1LG2 | 0 | 0 |
| PTP-1B | PTPN1 | 0 | 0 |
| Siglec-9 | SIGLEC9 | 0 | 0 |
| TGF-b R III | TGFBR3 | 0 | 0 |
| TSLP | TSLP | 0 | 0 |
| CTLA-4 | CTLA4 | 0 | 0 |
| a2-Antiplasmin | SERPINF2 | 0 | 0 |
| bFGF | FGF2 | 0 | 0 |
| Calpastatin | CAST | 0 | 0 |
| Ck-b-8-1 | CCL23 | 0 | 0 |
| DC-SIGN | CD209 | 0 | 0 |
| DC-SIGNR | CLEC4M | 0 | 1 |
| Ferritin | FTH1 FTL | 0 | 0 |
| FSH | CGA FSHB | 0 | 0 |
| Galectin-2 | LGALS2 | 0 | 0 |
| GFAP | GFAP | 0 | 0 |
| IL-19 | IL19 | 0 | 0 |
| IL-1b | IL1B | 0 | 1 |
| I-TAC | CXCL11 | 0 | 0 |
| MIP-1a | CCL3 | 0 | 0 |
| MRC2 | MRC2 | 0 | 0 |
| Myoglobin | MB | 0 | 0 |
| ON | SPARC | 0 | 0 |
| PARC | CCL18 | 0 | 0 |
| PTN | PTN | 0 | 0 |
| Resistin | RETN | 0 | 0 |
| Trypsin | PRSS1 | 0 | 0 |
| vWF | VWF | 1 | 0 |
| Fas ligand soluble | FASLG | 0 | 0 |
| Flt3 ligand | FLT3LG | 0 | 0 |
| Haptoglobin Mixed Type | HP | 0 | 0 |
| IL-4 sR | IL4R | 0 | 0 |
| NKG2D | KLRK1 | 0 | 0 |
| WISP-1 | WISP1 | 1 | 1 |
| BAFF | TNFSF13B | 0 | 0 |
| C9 | C9 | 0 | 0 |
| Cathepsin B | CTSB | 0 | 0 |
| FGF-5 | FGF5 | 0 | 0 |
| Galectin-3 | LGALS3 | 0 | 0 |
| GDF-9 | GDF9 | 0 | 0 |
| IgM | IGHM IGJ IGK@ IGL@ | 0 | 0 |
| IL-2 | IL2 | 0 | 0 |
| IL-13 | IL13 | 0 | 0 |
| IL-18BPa | IL18BP | 0 | 0 |
| LBP | LBP | 0 | 0 |
| Coagulation Factor Xa | F10 | 0 | 1 |
| P1GF | PGF | 0 | 0 |
| TIG2 | RARRES2 | 0 | 0 |
| ULBP-1 | ULBP1 | 0 | 0 |
| ULBP-2 | ULBP2 | 0 | 0 |
| XEDAR | EDA2R | 0 | 0 |
| Aurora kinase A | AURKA | 0 | 0 |
| DEAD-box protein 19B | DDX19B | 0 | 0 |
| MK01 | MAPK1 | 0 | 0 |
| SMAC | DIABLO | 1 | 1 |
| TRAIL R4 | TNFRSF10D | 0 | 0 |
| VEGF-C | VEGFC | 0 | 0 |
| sCD4 | CD4 | 0 | 0 |
| IL-2 sRa | IL2RA | 0 | 0 |
| TNF sR-II | TNFRSF1B | 0 | 0 |
| Siglec-3 | CD33 | 0 | 0 |
| ADAMTS-5 | ADAMTS5 | 0 | 0 |
| IDUA | IDUA | 0 | 0 |
| AMPM2 | METAP2 | 0 | 0 |
| amyloid precursor protein | APP | 0 | 0 |
| ARSB | ARSB | 0 | 0 |
| ASAHL | NAAA | 0 | 0 |
| ATS1 | ADAMTS1 | 0 | 0 |
| ATS13 | ADAMTS13 | 0 | 0 |
| Carbonic Anhydrase IV | CA4 | 0 | 0 |
| CATC | CTSC | 0 | 1 |
| Cathepsin A | CTSA | 0 | 0 |
| Cathepsin D | CTSD | 0 | 0 |
| Cathepsin S | CTSS | 0 | 0 |
| CD39 | ENTPD1 | 0 | 1 |
| Coagulation Factor VII | F7 | 0 | 0 |
| C2 | C2 | 0 | 0 |
| CRIS3 | CRISP3 | 0 | 1 |
| Enterokinase | PRSS7 | 0 | 0 |
| GAS1 | GAS1 | 0 | 1 |
| GASP-2 | GPRASP2 | 0 | 0 |
| Glutamate | CNDP2 | 0 | 0 |
| carboxypeptidase | | | |
| GPVI | GP6 | 0 | 0 |
| Granulysin | GNLY | 0 | 0 |
| HPLN1 | HAPLN1 | 0 | 0 |
| IDE | IDE | 0 | 0 |
| IDS | IDS | 0 | 0 |
| kallikrein 12 | KLK12 | 0 | 0 |
| kallikrein 13 | KLK13 | 0 | 0 |
| kallikrein 5 | KLK5 | 0 | 0 |
| KREM2 | KREMEN2 | 0 | 0 |
| LKHA4 | LTA4H | 0 | 0 |
| LYVE1 | LYVE1 | 0 | 0 |
| MATN3 | MATN3 | 0 | 1 |
| MEPE | MEPE | 0 | 0 |
| METAP1 | METAP1 | 0 | 0 |
| ASAH2 | ASAH2 | 0 | 0 |
| Nidogen | NID1 | 0 | 0 |
| NRP1 | NRP1 | 0 | 0 |
| PIGR | PIGR | 0 | 0 |
| Protease nexin I | SERPINE2 | 0 | 0 |
| PSMA | FOLH1 | 0 | 0 |
| RET | RET | 0 | 0 |
| SARP-2 | SFRP1 | 0 | 0 |
| Semaphorin 3A | SEMA3A | 0 | 0 |
| TrATPase | ACP5 | 0 | 0 |
| URB | CCDC80 | 0 | 0 |
| WFKN2 | WFIKKN2 | 0 | 0 |
| Aggrecan | ACAN | 0 | 0 |
| ANGL3 | ANGPTL3 | 0 | 0 |
| BGH3 | TGFBI | 0 | 0 |
| BGN | BGN | 0 | 0 |
| C1r | C1R | 0 | 0 |
| Carbonic Anhydrase X | CA10 | 0 | 0 |
| CD109 | CD109 | 0 | 0 |
| CD23 | FCER2 | 0 | 0 |
| CD48 | CD48 | 0 | 0 |
| CD5L | CD5L | 0 | 0 |
| CFC1 | CFC1 | 0 | 0 |
| CNTN2 | CNTN2 | 0 | 0 |
| Contactin-4 | CNTN4 | 0 | 0 |
| Contactin-5 | CNTN5 | 0 | 0 |
| CYTF | CST7 | 0 | 0 |
| Cystatin M | CST6 | 0 | 0 |
| DLL4 | DLL4 | 0 | 1 |
| FCG2A/B | FCGR2A FCGR2B | 0 | 0 |
| FCG3B | FCGR3B | 0 | 0 |
| FCGR1 | FCGR1A | 0 | 0 |
| FCN2 | FCN2 | 0 | 0 |
| GFRa-1 | GFRA1 | 0 | 0 |
| GPC2 | GPC2 | 0 | 0 |
| Heparin cofactor II | SERPIND1 | 0 | 0 |
| HTRA2 | HTRA2 | 0 | 0 |
| IGFBP-7 | IGFBP7 | 0 | 0 |
| IL24 | IL24 | 0 | 0 |
| LRIG3 | LRIG3 | 0 | 0 |
| LRP8 | LRP8 | 0 | 0 |
| LY9 | LY9 | 0 | 0 |
| MATN2 | MATN2 | 0 | 0 |
| Nectin-like protein 2 | CADM1 | 0 | 0 |
| NET4 | NTN4 | 0 | 0 |
| PGRP-S | PGLYRP1 | 0 | 0 |
| RGMB | RGMB | 0 | 0 |
| RGM-C | HFE2 | 0 | 0 |
| TFPI | TFPI | 0 | 0 |
| TSP2 | THBS2 | 0 | 0 |
| TSP4 | THBS4 | 0 | 0 |
| ABL1 | ABL1 | 0 | 0 |
| Aminoacylase-1 | ACY1 | 0 | 0 |
| Antithrombin III | SERPINC1 | 0 | 0 |
| AURKB | AURKB | 0 | 0 |
| BARK1 | ADRBK1 | 0 | 0 |
| BMP-1 | BMP1 | 0 | 0 |
| CAMK2A | CAMK2A | 0 | 0 |
| CAMK2B | CAMK2B | 0 | 0 |
| Carbonic anhydrase 6 | CA6 | 0 | 0 |
| Carbonic anhydrase VII | CA7 | 0 | 0 |
| CDK2/cyclin A | CDK2 CCNA2 | 0 | 0 |
| CDK5/p35 | CDK5 CDK5R1 | 0 | 0 |
| CDK8/cyclin C | CDK8 CCNC | 0 | 0 |
| Chk2 | CHEK2 | 0 | 0 |
| CLC4K | CD207 | 0 | 0 |
| CRDL1 | CHRDL1 | 0 | 0 |
| CSK | CSK | 0 | 0 |
| Cathepsin V | CTSL2 | 0 | 0 |
| Dkk-4 | DKK4 | 0 | 0 |
| ECM1 | ECM1 | 0 | 0 |
| FETUB | FETUB | 0 | 0 |
| Granzyme H | GZMH | 0 | 0 |
| HCK | HCK | 0 | 0 |
| IL-17 RD | IL17RD | 0 | 0 |
| Kallikrein 7 | KLK7 | 0 | 0 |
| KPCI | PRKCI | 0 | 0 |
| LYNB | LYN | 0 | 0 |
| PAK3 | PAK3 | 0 | 0 |
| PAK7 | PAK7 | 0 | 0 |
| PCI | SERPINA5 | 0 | 1 |
| PIK3CA/PIK3R1 | PIK3CA PIK3R1 | 0 | 0 |
| PK3CG | PIK3CG | 0 | 1 |
| PKB a/b/g | None | 0 | 0 |
| PLK-1 | PLK1 | 0 | 0 |
| Renin | REN | 0 | 0 |
| SHP-2 | PTPN11 | 0 | 0 |
| STAB2 | STAB2 | 0 | 1 |
| TBK1 | TBK1 | 0 | 0 |
| TCPTP | PTPN2 | 0 | 0 |
| TPSB2 | TPSB2 | 0 | 0 |
| TPSG1 | TPSG1 | 0 | 0 |
| UFC1 | UFC1 | 0 | 0 |
| Bcl-2 | BCL2 | 0 | 0 |
| BFL1 | BCL2A1 | 0 | 0 |
| BMX | BMX | 0 | 0 |
| BSP | IBSP | 0 | 0 |
| BTK | BTK | 0 | 0 |
| CAMK1D | CAMK1D | 0 | 1 |
| CAMK2D | CAMK2D | 0 | 0 |
| Carbonic anhydrase XIII | CA13 | 0 | 0 |
| CD30 Ligand | TNFSF8 | 0 | 0 |
| CDK1/cyclin B | CDC2 CCNB1 | 0 | 0 |
| Chymase | CMA1 | 0 | 1 |
| CSK21 | CSNK2A1 | 0 | 0 |
| EphA1 | EPHA1 | 0 | 0 |
| EPHA3 | EPHA3 | 0 | 0 |
| FN1.3 | FN1 | 1 | 0 |
| FN1.4 | FN1 | 0 | 0 |
| Flt-3 | FLT3 | 0 | 0 |
| FSTL3 | FSTL3 | 0 | 0 |
| granzyme A | GZMA | 0 | 0 |
| GSK-3 alpha/beta | GSK3A GSK3B | 0 | 0 |
| HIPK3 | HIPK3 | 0 | 0 |
| IL-15 Ra | IL15RA | 0 | 0 |
| IL-18 Ra | IL18R1 | 0 | 0 |
| IL-8 | IL8 | 0 | 0 |
| IR | INSR | 0 | 0 |
| Kallistatin | SERPINA4 | 0 | 0 |
| Kallikrein 6 | KLK6 | 0 | 0 |
| LCK | LCK | 0 | 0 |
| LYN | LYN | 0 | 0 |
| Periostin | POSTN | 0 | 0 |
| PDGF Rb | PDGFRB | 0 | 0 |
| PGCB | BCAN | 0 | 0 |
| PRKACA | PRKACA | 0 | 0 |
| RPS6KA3 | RPS6KA3 | 0 | 0 |
| sE-Selectin | SELE | 0 | 0 |
| STK16 | STK16 | 0 | 1 |
| Survivin | BIRC5 | 0 | 1 |
| Thrombopoietin Receptor | MPL | 0 | 0 |
| Thrombospondin-1 | THBS1 | 0 | 0 |
| TrkA | NTRK1 | 0 | 0 |
| TRY3 | PRSS3 | 0 | 0 |
| DUS3 | DUSP3 | 0 | 0 |
| XPNPEP1 | XPNPEP1 | 0 | 0 |
| Angiotensinogen | AGT | 0 | 0 |
| b2-Microglobulin | B2M | 0 | 0 |
| b-ECGF | FGF1 | 0 | 0 |
| BLC | CXCL13 | 0 | 0 |
| Catalase | CAT | 0 | 0 |
| CNTF | CNTF | 0 | 0 |
| Epo | EPO | 1 | 0 |
| FGF-17 | FGF17 | 1 | 1 |
| GCP-2 | CXCL6 | 0 | 0 |
| IFN-aA | IFNA2 | 0 | 1 |
| IL-17 | IL17A | 0 | 0 |
| IL-17B | IL17B | 0 | 0 |
| Integrin a1b1 | ITGA1 ITGB1 | 0 | 0 |
| LEAP-1 | HAMP | 0 | 0 |
| Lymphotoxin a1/b2 | LTA LTB | 0 | 0 |
| Lymphotoxin a2/b1 | LTA LTB | 0 | 0 |
| MDC | CCL22 | 0 | 0 |
| MIP-5 | CCL15 | 0 | 0 |
| Proteinase-3 | PRTN3 | 0 | 0 |
| SDF-1 | CXCL12 | 0 | 0 |
| TAFI | CPB2 | 0 | 0 |
| TARC | CCL17 | 0 | 0 |
| TGF-b3 | TGFB3 | 1 | 1 |
| TSH | CGA TSHB | 0 | 0 |
| Vasoactive Intestinal | VIP | 0 | 0 |
| Peptide | | | |
| CD40 ligand soluble | CD40LG | 0 | 0 |
| DKK1 | DKK1 | 0 | 0 |
| dopa decarboxylase | DDC | 0 | 0 |
| Adiponectin | ADIPOQ | 0 | 0 |
| a1-Antitrypsin | SERPINA1 | 0 | 0 |
| a2-HS-Glycoprotein | AHSG | 0 | 0 |
| Arylsulfatase A | ARSA | 0 | 0 |
| BASI | BSG | 0 | 1 |
| BMP10 | BMP10 | 0 | 1 |
| C1s | C1S | 0 | 0 |
| Cadherin-6 | CDH6 | 0 | 1 |
| CAMK1 | CAMK1 | 0 | 1 |
| Caspase-3 | CASP3 | 0 | 0 |
| CATE | CTSE | 0 | 0 |
| Chitotriosidase-1 | CHIT1 | 0 | 0 |
| CHL1 | CHL1 | 0 | 0 |
| CLC7A | CLEC7A | 0 | 1 |
| CNDP1 | CNDP1 | 0 | 0 |
| MASP3 | MASP1 | 0 | 0 |
| Discoidin domain | DDR2 | 0 | 1 |
| receptor 2 | | | |
| DKK3 | DKK3 | 0 | 0 |
| DPP2 | DPP7 | 0 | 1 |
| Endothelin-converting | ECE1 | 0 | 0 |
| enzyme 1 | | | |
| EphB4 | EPHB4 | 0 | 1 |
| FCN1 | FCN1 | 0 | 0 |
| GNS | GNS | 0 | 0 |
| HGFA | HGFAC | 0 | 0 |
| IL22RA1 | IL22RA1 | 0 | 0 |
| LGMN | LGMN | 0 | 1 |
| LY86 | LY86 | 0 | 0 |
| Marapsin | PRSS27 | 0 | 1 |
| MMEL2 | MMEL1 | 0 | 1 |
| MP2K2 | MAP2K2 | 0 | 0 |
| MRCKB | CDC42BPB | 0 | 1 |
| Nectin-like protein 1 | CADM3 | 0 | 1 |
| NID2 | NID2 | 0 | 0 |
| OBCAM | OPCML | 0 | 1 |
| OCAD1 | OCIAD1 | 0 | 0 |
| OLR1 | OLR1 | 0 | 0 |
| RAP | LRPAP1 | 0 | 0 |
| RBP | RBP4 | 0 | 0 |
| SLAF5 | CD84 | 0 | 1 |
| Soggy-1 | DKKL1 | 0 | 1 |
| TEC | TEC | 0 | 0 |
| TLR4:MD-2 complex | TLR4 LY96 | 0 | 0 |
| VEGF sR2 | KDR | 0 | 0 |
| BMPER | BMPER | 0 | 0 |
| Cadherin-12 | CDH12 | 0 | 1 |
| Calcineurin B a | PPP3R1 | 0 | 1 |
| COLEC12 | COLEC12 | 0 | 1 |
| complement factor H- | CFHR5 | 0 | 0 |
| related 5 | | | |
| IGF-II receptor | IGF2R | 0 | 0 |
| kallikrein 14 | KLK14 | 0 | 1 |
| Macrophage scavenger | MSR1 | 0 | 1 |
| receptor | | | |
| MFRP | MFRP | 0 | 0 |
| IgG | IGHG1 IGHG2 IGHG3 | 0 | 0 |
| | IGHG4 IGK@ IGL@ | | |
| Albumin | ALB | 0 | 0 |
| a2-Macroglobulin | A2M | 0 | 0 |
| ALT | GPT | 0 | 0 |
| Angiostatin | PLG | 0 | 0 |
| CK-MB | CKB CKM | 0 | 0 |
| IFN-g R1 | IFNGR1 | 0 | 0 |
| p27Kip1 | CDKN1B | 0 | 0 |
| TNF-a | TNF | 0 | 0 |
| BNP-32 | NPPB | 0 | 0 |
| PTH | PTH | 0 | 0 |
| PYY | PYY | 0 | 0 |
| Secretin | SCT | 0 | 0 |
| Somatostatin-28 | SST | 0 | 0 |
| TNR4 | TNFRSF4 | 0 | 0 |
| BMP-6 | BMP6 | 0 | 1 |
| Cathepsin H | CTSH | 0 | 0 |
| CSF-1 | CSF1 | 0 | 0 |
| gpIIbIIIa | ITGA2B ITGB3 | 0 | 0 |
| IL-5 | IL5 | 0 | 1 |
| MMP-10 | MMP10 | 0 | 0 |
| Activated Protein C | PROC | 0 | 0 |
| COX-2 | PTGS2 | 0 | 0 |
| STX1a | STX1A | 0 | 0 |
| sTie-2 | TEK | 0 | 0 |
| ADAM 9 | ADAM9 | 0 | 0 |
| ANGL4 | ANGPTL4 | 0 | 0 |
| Cadherin-2 | CDH2 | 0 | 0 |
| Carbonic anhydrase 9 | CA9 | 0 | 0 |
| Carbonic anhydrase III | CA3 | 0 | 0 |
| CK-BB | CKB | 0 | 0 |
| Cystatin-S | CST4 | 0 | 1 |
| CYTD | CST5 | 0 | 0 |
| DMP1 | DMP1 | 0 | 0 |
| Endocan | ESM1 | 0 | 0 |
| EphA5 | EPHA5 | 0 | 0 |
| FGF23 | FGF23 | 1 | 0 |
| FGFR-2 | FGFR2 | 0 | 0 |
| FGFR-3 | FGFR3 | 0 | 0 |
| FGR | FGR | 0 | 0 |
| Ficolin-3 | FCN3 | 0 | 0 |
| FYN | FYN | 0 | 0 |
| IL-11 RA | IL11RA | 0 | 0 |
| IL-12 RB2 | IL12RB2 | 0 | 0 |
| KPCT | PRKCQ | 0 | 0 |
| MAPK2 | MAPKAPK2 | 0 | 0 |
| MAPK5 | MAPKAPK5 | 0 | 0 |
| MAPKAPK3 | MAPKAPK3 | 0 | 0 |
| MATK | MATK | 0 | 0 |
| MK08 | MAPK8 | 0 | 0 |
| PAK6 | PAK6 | 1 | 0 |
| PDGF-CC | PDGFC | 0 | 0 |
| pTEN | PTEN | 0 | 0 |
| PTK6 | PTK6 | 0 | 0 |
| RGMA | RGMA | 0 | 0 |
| TLR2 | TLR2 | 0 | 1 |
| UFM1 | UFM1 | 0 | 0 |
| AIP | AIP | 0 | 0 |
| Cyclophilin A | PPIA | 0 | 0 |
| DLRB1 | DYNLRB1 | 0 | 0 |
| ETHE1 | ETHE1 | 0 | 0 |
| GAPDH liver | GAPDH | 0 | 0 |
| HSP 40 | DNAJB1 | 0 | 0 |
| MDHC | MDH1 | 0 | 0 |
| NACA | NACA | 0 | 0 |
| Peroxiredoxin-1 | PRDX1 | 0 | 0 |
| PPAC | ACP1 | 0 | 0 |
| PSA1 | PSMA1 | 0 | 0 |
| PSA6 | PSMA6 | 0 | 0 |
| RS7 | RPS7 | 0 | 0 |
| RSK-like protein kinase | RPS6KA5 | 0 | 0 |
| SBDS | SBDS | 0 | 0 |
| SE6L2 | SEZ6L2 | 0 | 0 |
| SGTA | SGTA | 0 | 0 |
| TCTP | TPT1 | 0 | 0 |
| TMA | TPO | 0 | 1 |
| UB2L3 | UBE2L3 | 0 | 0 |
| ARI3A | ARID3A | 0 | 0 |
| ASGR1 | ASGR1 | 0 | 0 |
| CaMKK alpha | CAMKK1 | 0 | 0 |
| CDC37 | CDC37 | 0 | 0 |
| DLC8 | DYNLL1 | 0 | 0 |
| EF-1-beta | EEF1B2 | 0 | 0 |
| eIF-5A-l | EIF5A | 0 | 0 |
| HINT1 | HINT1 | 0 | 0 |
| IMB1 | KPNB1 | 0 | 0 |
| ING1 | ING1 | 0 | 0 |
| Lamin-B1 | LMNB1 | 0 | 0 |
| LDH-H 1 | LDHB | 0 | 0 |
| MBD4 | MBD4 | 0 | 0 |
| MED-1 | MED1 | 0 | 0 |
| Mesothelin | MSLN | 0 | 0 |
| NAGK | NAGK | 0 | 0 |
| Phosphoglycerate mutase 1 | PGAM1 | 0 | 0 |
| PLPP | PDXP | 0 | 0 |
| PSD7 | PSMD7 | 0 | 0 |
| SKP1 | SKP1 | 0 | 0 |
| Sorting nexin 4 | SNX4 | 0 | 0 |
| UBE2N | UBE2N | 0 | 0 |
| discoidin domain receptor 1 | DDR1 | 0 | 0 |
| FGF-4 | FGF4 | 0 | 0 |
| HSP 70 | HSPA1A | 0 | 0 |
| sRAGE | AGER | 0 | 0 |
| BPI | BPI | 0 | 0 |
| C6 | C6 | 0 | 1 |
| Eotaxin-2 | CCL24 | 0 | 1 |
| Factor B | CFB | 0 | 0 |
| FGF-6 | FGF6 | 0 | 0 |
| Fibronectin | FN1 | 1 | 0 |
| FST | FST | 0 | 0 |
| Granzyme B | GZMB | 0 | 0 |
| HB-EGF | HBEGF | 1 | 1 |
| IgE | IGHE IGK@ IGL@ | 0 | 0 |
| IL-17D | IL17D | 0 | 0 |
| IL-17E | IL25 | 0 | 0 |
| IL-20 | IL20 | 0 | 0 |
| IL-6 sRa | IL6R | 0 | 0 |
| IL-7 | IL7 | 0 | 0 |
| IP-10 | CXCL10 | 0 | 0 |
| Lymphotactin | XCL1 | 0 | 0 |
| MCP-4 | CCL13 | 0 | 0 |
| Neurotrophin-3 | NTF3 | 0 | 0 |
| Neurotrophin-5 | NTF4 | 0 | 0 |
| PAPP-A | PAPPA | 0 | 0 |
| PDGF-BB | PDGFB | 0 | 0 |
| Plasmin | PLG | 0 | 0 |
| Plasminogen | PLG | 0 | 0 |
| Prekallikrein | KLKB1 | 0 | 0 |
| PSA-ACT | KLK3 SERPINA3 | 0 | 0 |
| P-Selectin | SELP | 0 | 0 |
| Tenascin | TNC | 0 | 0 |
| TGF-b2 | TGFB2 | 0 | 0 |
| Thrombin | F2 | 0 | 0 |
| uPA | PLAU | 0 | 0 |
| Factor H | CFH | 0 | 1 |
| MMP-2 | MMP2 | 0 | 0 |
| Transferrin | TF | 0 | 1 |
| Histone H2A.z | H2AFZ | 0 | 0 |
| Thyroglobulin | TG | 0 | 0 |
| 14-3-3 | YWHAB YWHAE | 0 | 0 |
| | YWHAG YWHAH WHAQ | | |
| | YWHAZ SFN | | |
| 4EBP2 | EIF4EBP2 | 0 | 0 |
| 6-Phosphogluconate | PGD | 0 | 0 |
| dehydrogenase | | | |
| Aflatoxin B1 aldehyde | AKR7A2 | 0 | 0 |
| reductase | | | |
| AK1A1 | AKR1A1 | 0 | 0 |
| AN32B | ANP32B | 0 | 0 |
| Cofilin-1 | CFL1 | 0 | 0 |
| DRG-1 | VTA1 | 0 | 0 |
| Dynactin subunit 2 | DCTN2 | 0 | 0 |
| EP15R | EPS15L1 | 0 | 0 |
| ERAB | HSD17B10 | 1 | 0 |
| FER | FER | 0 | 0 |
| HNRPQ | SYNCRIP | 0 | 0 |
| HSP70 protein 8 | HSPA8 | 1 | 0 |
| IF4G2 | EIF4G2 | 1 | 0 |
| IGF-I sR | IGF1R | 0 | 0 |
| IL-1 R4 | IL1RL1 | 0 | 0 |
| LCMT1 | LCMT1 | 0 | 0 |
| LIN7B | LIN7B | 0 | 0 |
| M2-PK | PKM2 | 0 | 0 |
| MDM2 | MDM2 | 0 | 0 |
| NCAM-L1 | L1CAM | 0 | 0 |
| NDP kinase B | NME2 | 0 | 0 |
| NSF1C | NSFL1C | 0 | 0 |
| Nucleoside diphosphate | NME1 | 1 | 0 |
| kinase A | | | |
| NUDC3 | NUDCD3 | 0 | 0 |
| PA2G4 | PA2G4 | 0 | 0 |
| paraoxonase 1 | PON1 | 0 | 0 |
| PESC | PES1 | 0 | 0 |
| PFD5 | PFDN5 | 0 | 0 |
| PHI | GPI | 0 | 0 |
| prostatic binding protein | PEBP1 | 0 | 0 |
| Protein disulfide-isomerase | P4HB | 0 | 0 |
| PSA2 | PSMA2 | 0 | 0 |
| RAN | RAN | 0 | 0 |
| RBM39 | RBM39 | 0 | 0 |
| SNAA | NAPA | 0 | 0 |
| Sphingosine kinase 1 | SPHK1 | 0 | 0 |
| Spondin-1 | SPON1 | 0 | 0 |
| Thymidine kinase | TK1 | 0 | 0 |
| transcription factor MLR1 | LCORL | 0 | 0 |
| isoform CRA_b | | | |
| Transketolase | TKT | 0 | 0 |
| Triosephosphate isomerase | TPI1 | 0 | 0 |
| XTP3A | DCTPP1 | 0 | 0 |
| PTP-1C | PTPN6 | 0 | 0 |
| AMNLS | AMN | 0 | 0 |
| CYTT | CST2 | 0 | 0 |
| BOC | BOC | 0 | 0 |
| PSA | KLK3 | 0 | 0 |
| CLC1B | CLEC1B | 0 | 0 |
| SAA | SAA1 | 0 | 0 |
| CRP | CRP | 0 | 0 |
| sICAM-1 | ICAM1 | 0 | 0 |
| DAPK2 | DAPK2 | 0 | 0 |
| DYRK3 | DYRK3 | 0 | 1 |
| b-NGF | NGF | 0 | 0 |
| Activin AB | INHBA INHBB | 0 | 0 |
| DHH | DHH | 0 | 0 |
| FGF-12 | FGF12 | 0 | 0 |
| FGF-16 | FGF16 | 0 | 0 |
| FGF-8A | FGF8 | 0 | 0 |
| IFN-lambda 1 | IL29 | 0 | 0 |
| IFN-lambda 2 | IL28A | 0 | 0 |
| MSP | MST1 | 0 | 0 |
| SLPI | SLPI | 0 | 0 |
| SP-D | SFTPD | 0 | 0 |
| ADAM12 | ADAM12 | 0 | 0 |
| BCL2-like 1 protein | BCL2L1 | 0 | 0 |
| CHST2 | CHST2 | 1 | 0 |
| CHST6 | CHST6 | 0 | 0 |
| Collectin Kidney 1 | COLEC11 | 0 | 0 |
| ENPP7 | ENPP7 | 0 | 0 |
| ENTP3 | ENTPD3 | 0 | 1 |
| ENTP5 | ENTPD5 | 0 | 0 |
| FCRL3 | FCRL3 | 0 | 0 |
| GREM1 | GREM1 | 1 | 0 |
| hnRNP A/B | HNRNPAB | 0 | 0 |
| LRRT1 | LRRTM1 | 0 | 0 |
| LRRT3 | LRRTM3 | 0 | 0 |
| MFGM | MFGE8 | 0 | 0 |
| PCSK7 | PCSK7 | 0 | 0 |
| PDPK1 | PDPK1 | 0 | 0 |
| RASA1 | RASA1 | 0 | 1 |
| Sialoadhesin | SIGLEC1 | 0 | 0 |
| SPARCL1 | SPARCL1 | 0 | 0 |
| SPHK2 | SPHK2 | 0 | 0 |
| ST4S6 | CHST15 | 0 | 0 |
| TGM3 | TGM3 | 1 | 0 |
| Tropomyosin 2 | TPM2 | 0 | 0 |
| Ubiquitin | RPS27A | 1 | 0 |
| ZAP70 | ZAP70 | 0 | 0 |
| C1-Esterase Inhibitor | SERPING1 | 0 | 0 |
| C3b | C3 | 1 | 0 |
| C4 | C4A C4B | 0 | 1 |
| C5b 6 Complex | C5 C6 | 0 | 0 |
| FGF7 | FGF7 | 0 | 0 |
| IL-3 Ra | IL3RA | 0 | 0 |
| IL-5 Ra | IL5RA | 0 | 0 |
| IL-11 | IL11 | 0 | 0 |
| IL-23 | IL12B IL23A | 0 | 0 |
| Kininogen HMW | KNG1 | 0 | 0 |
| MMP-12 | MMP12 | 0 | 0 |
| NCAM-120 | NCAM1 | 0 | 0 |
| PDGF-AA | PDGFA | 0 | 0 |
| SCGF-alpha | CLEC11A | 0 | 0 |
| ATS15 | ADAMTS15 | 0 | 0 |
| BSSP4 | PRSS22 | 0 | 0 |
| BST1 | BST1 | 0 | 0 |
| CBX5 | CBX5 | 0 | 1 |
| CDON | CDON | 0 | 0 |
| Clusterin | CLU | 0 | 1 |
| CONA1 | COL23A1 | 0 | 0 |
| CTAP-III | PPBP | 0 | 0 |
| DnaJ homolog | DNAJC19 | 0 | 0 |
| EMR2 | EMR2 | 0 | 0 |
| FLRT1 | FLRT1 | 0 | 0 |
| Fucosyltransferase 3 | FUT3 | 0 | 0 |
| FUT5 | FUT5 | 0 | 0 |
| GP114 | GPR114 | 0 | 1 |
| H6ST1 | HS6ST1 | 0 | 0 |
| HDGR2 | HDGFRP2 | 0 | 0 |
| IL-34 | IL34 | 0 | 0 |
| KIRR3 | KIRREL3 | 0 | 0 |
| KYNU | KYNU | 0 | 0 |
| Livin B | BIRC7 | 0 | 1 |
| NXPH1 | NXPH1 | 0 | 0 |
| PLCG1 | PLCG1 | 0 | 0 |
| PLXC1 | PLXNC1 | 0 | 0 |
| RSPO2 | RSPO2 | 0 | 0 |
| SH21A | SH2D1A | 0 | 0 |
| SLIK5 | SLITRK5 | 0 | 0 |
| SORC2 | SORCS2 | 0 | 0 |
| PH | PPY | 0 | 0 |
| PACAP-27 | ADCYAP1 | 0 | 1 |
| PACAP-38 | ADCYAP1 | 0 | 0 |
| IL-6 | IL6 | 0 | 0 |
| 3HIDH | HIBADH | 0 | 0 |
| CASA | CSN1S1 | 1 | 1 |
| FABP | FABP3 | 0 | 0 |
| GM-CSF | CSF2 | 0 | 0 |
| TNF-b | LTA | 0 | 0 |
| 41 | EPB41 | 0 | 0 |
| 17-beta-HSD 1 | HSD17B1 | 0 | 1 |
| ApoD | APOD | 0 | 0 |
| IL-3 | IL3 | 0 | 0 |
| PPIB | PPIB | 0 | 1 |
| Protein disulfide isomerase | PDIA3 | 0 | 0 |
| A3 | | | |
| TFF3 | TFF3 | 0 | 0 |
| Afamin | AFM | 0 | 0 |
| Olfactomedin-4 | OLFM4 | 0 | 0 |
| ASM3A | SMPDL3A | 0 | 0 |
| FAM107B | FAM107B | 0 | 0 |
| Gelsolin | GSN | 0 | 0 |
| CBG | SERPINA6 | 0 | 1 |
| Cytidylate kinase | CMPK1 | 0 | 1 |
| C34 gp41 HIV Fragment | Human-virus | 0 | 0 |
| PERL | LPO | 0 | 0 |
| CO8A1 | COL8A1 | 0 | 0 |
| ITI heavy chain H4 | ITIH4 | 0 | 0 |
| TXD12 | TXNDC12 | 0 | 0 |
| STRATIFIN | SFN | 0 | 0 |
| sL-Selectin | SELL | 0 | 0 |
| TRAIL R1 | TNFRSF10A | 0 | 1 |
| Epithelial cell kinase | EPHA2 | 0 | 0 |
| G-CSF | CSF3 | 1 | 1 |
| Glypican 3 | GPC3 | 0 | 0 |
| IL-1a | IL1A | 0 | 0 |
| BMPR1A | BMPR1A | 0 | 0 |
| BMP RII | BMPR2 | 0 | 0 |
| TrkB | NTRK2 | 0 | 0 |
| VEGF121 | VEGFA | 0 | 0 |
| Angiogenin | ANG | 0 | 0 |
| C3d | C3 | 0 | 0 |
| Coagulation Factor IX | F9 | 0 | 0 |
| Coagulation Factor X | F10 | 0 | 0 |
| GDF2 | GDF2 | 0 | 0 |
| Insulin | INS | 0 | 0 |
| MCP-3 | CCL7 | 0 | 0 |
| WNT7A | WNT7A | 0 | 0 |
| ACTH | POMC | 0 | 0 |
| Glucagon | GCG | 0 | 0 |
| C3a | C3 | 1 | 1 |
| Calcineurin | PPP3CA PPP3R1 | 0 | 0 |
| Caspase-2 | CASP2 | 0 | 0 |
| Coactosin-like protein | COTL1 | 0 | 1 |
| Coagulation Factor V | F5 | 0 | 1 |
| D-dimer | FGA FGB FGG | 0 | 0 |
| Endoglin | ENG | 0 | 0 |
| Galectin-8 | LGALS8 | 0 | 0 |
| GIB | PLA2G1B | 0 | 0 |
| Glutathione S-transferase Pi | GSTP1 | 0 | 0 |
| GOT1 | GOT1 | 0 | 0 |
| HCC-4 | CCL16 | 0 | 0 |
| HCG | CGA CGB | 0 | 0 |
| Hemoglobin | HBA1 HBB | 1 | 0 |
| IgD | IGHD IGK@ IGL@ | 0 | 0 |
| Integrin aVb5 | ITGAV ITGB5 | 0 | 0 |
| LIF sR | LIFR | 0 | 0 |
| Lysozyme | LYZ | 0 | 0 |
| MIP-3b | CCL19 | 0 | 0 |
| MIS | AMH | 0 | 0 |
| MMP-1 | MMP1 | 0 | 0 |
| MMP-13 | MMP13 | 0 | 0 |
| SHBG | SHBG | 0 | 0 |
| Stanniocalcin-1 | STC1 | 0 | 0 |
| TF | F3 | 0 | 0 |
| EPI | EREG | 0 | 0 |
| 40S ribosomal protein SA | RPSA | 0 | 1 |
| AGR2 | AGR2 | 0 | 1 |
| annexin I | ANXA1 | 0 | 0 |
| annexin II | ANXA2 | 0 | 0 |
| ARMEL | CDNF | 0 | 0 |
| ARP19 | ARPP19 | 0 | 0 |
| ARTS1 | ERAP1 | 0 | 0 |
| ATP synthase beta chain | ATP5B | 0 | 1 |
| C1QBP | C1QBP | 0 | 1 |
| CAPG | CAPG | 0 | 0 |
| Carbonic anhydrase I | CA1 | 0 | 0 |
| carbonic anhydrase II | CA2 | 0 | 0 |
| CATZ | CTSZ | 0 | 0 |
| cIAP-2 | BIRC3 | 0 | 0 |
| CRK | CRK | 0 | 0 |
| DBNL | DBNL | 0 | 0 |
| DERM | DPT | 0 | 0 |
| DSC3 | DSC3 | 0 | 0 |
| Elafin | PI3 | 0 | 0 |
| ERP29 | ERP29 | 1 | 0 |
| Esterase D | ESD | 0 | 0 |
| FABPE | FABP5 | 0 | 0 |
| FAK1 | PTK2 | 0 | 0 |
| FCAR | FCAR | 0 | 0 |
| FGFR4 | FGFR4 | 0 | 0 |
| Fibrinogen g-chain dimer | FGG | 0 | 0 |
| GP1BA | GP1BA | 0 | 0 |
| GPC5 | GPC5 | 0 | 0 |
| GRN | GRN | 0 | 0 |
| GSTA3 | GSTA3 | 1 | 0 |
| hnRNP K | HNRNPK | 0 | 0 |
| HPG- | HPGD | 0 | 0 |
| HRG | HRG | 0 | 0 |
| IF4A3 | EIF4A3 | 0 | 0 |
| JAK2 | JAK2 | 0 | 0 |
| LG3BP | LGALS3BP | 0 | 0 |
| Mammaglobin 2 | SCGB2A1 | 0 | 1 |
| MMP-14 | MMP14 | 0 | 0 |
| MK11 | MAPK11 | 0 | 0 |
| MK12 | MAPK12 | 0 | 0 |
| MK13 | MAPK13 | 0 | 0 |
| MAPK14 | MAPK14 | 0 | 0 |
| Mn SOD | SOD2 | 0 | 0 |
| Moesin | MSN | 0 | 0 |
| PBEF | NAMPT | 0 | 0 |
| Myokinase human | AK1 | 0 | 0 |
| NCC27 | CLIC1 | 0 | 0 |
| NCK1 | NCK1 | 0 | 0 |
| PAFAH | PLA2G7 | 0 | 0 |
| PARK7 | PARK7 | 0 | 0 |
| Peroxiredoxin-5 | PRDX5 | 0 | 1 |
| Peroxiredoxin-6 | PRDX6 | 0 | 0 |
| PGP9.5 | UCHL1 | 0 | 0 |
| phosphoglycerate kinase 1 | PGK1 | 0 | 0 |
| PPase | PPA1 | 0 | 0 |
| PSME1 | PSME1 | 0 | 0 |
| PUR8 | ADSL | 1 | 1 |
| Rb | RB1 | 0 | 0 |
| RS3 | RPS3 | 0 | 0 |
| sCD163 | CD163 | 0 | 0 |
| SEPR | FAP | 0 | 0 |
| SIRT2 | SIRT2 | 0 | 1 |
| SPTA2 | SPTAN1 | 0 | 0 |
| SSRP1 | SSRP1 | 0 | 0 |
| Tropomyosin 1 alpha chain | TPM1 | 0 | 0 |
| Trypsin 2 | PRSS2 | 0 | 0 |
| TS | TYMS | 0 | 0 |
| TSG-6 | TNFAIP6 | 0 | 0 |
| AMHR2 | AMHR2 | 0 | 0 |
| B7-H1 | CD274 | 0 | 0 |
| B7-H2 | ICOSLG | 0 | 0 |
| CD226 | CD226 | 0 | 0 |
| CLM6 | CD300C | 0 | 0 |
| CRTAM | CRTAM | 0 | 0 |
| DAF | CD55 | 0 | 0 |
| DcR3 | TNFRSF6B | 0 | 0 |
| Desmoglein-2 | DSG2 | 0 | 0 |
| DR3 | TNFRSF25 | 0 | 0 |
| EPHAA | EPHA10 | 0 | 0 |
| EPHB2 | EPHB2 | 0 | 0 |
| EphB6 | EPHB6 | 0 | 0 |
| GITR | TNFRSF18 | 0 | 0 |
| GPNMB | GPNMB | 0 | 0 |
| IL-1 sR9 | IL1RAPL2 | 0 | 1 |
| IL-17B R | IL17RB | 0 | 0 |
| IL-20 Ra | IL20RA | 0 | 0 |
| IL-22BP | IL22RA2 | 0 | 0 |
| IL-23 R | IL23R | 0 | 0 |
| IL-7 Ra | IL7R | 0 | 0 |
| ILT-2 | LILRB1 | 0 | 0 |
| ILT-4 | LILRB2 | 0 | 0 |
| JAG1 | JAG1 | 0 | 0 |
| JAG2 | JAG2 | 0 | 0 |
| JAML1 | AMICA1 | 0 | 0 |
| KI2L4 | KIR2DL4 | 0 | 0 |
| KI3L2 | KIR3DL2 | 0 | 0 |
| KI3S1 | KIR3DS1 | 0 | 0 |
| KLRF1 | KLRF1 | 0 | 0 |
| LAG-3 | LAG3 | 0 | 0 |
| LIMP II | SCARB2 | 0 | 0 |
| MICB | MICB | 0 | 0 |
| MO2R1 | CD200R1 | 0 | 0 |
| NKp46 | NCR1 | 0 | 0 |
| Nogo Receptor | RTN4R | 0 | 0 |
| NOTC2 | NOTCH2 | 0 | 0 |
| Notch 1 | NOTCH1 | 0 | 0 |
| Notch-3 | NOTCH3 | 0 | 0 |
| Nr-CAM | NRCAM | 0 | 0 |
| NRX1B | NRXN1 | 0 | 0 |
| NRX3B | NRXN3 | 0 | 0 |
| OX2G | CD200 | 0 | 0 |
| Prolactin Receptor | PRLR | 0 | 0 |
| RELT | RELT | 0 | 0 |
| ROBO2 | ROBO2 | 0 | 0 |
| ROBO3 | ROBO3 | 0 | 0 |
| RTN4 | RTN4 | 0 | 0 |
| Semaphorin-6A | SEMA6A | 0 | 0 |
| sICAM-5 | ICAM5 | 0 | 0 |
| SIG14 | SIGLEC14 | 0 | 0 |
| SLAF6 | SLAMF6 | 0 | 0 |
| SREC-I | SCARF1 | 0 | 0 |
| SREC-II | SCARF2 | 0 | 0 |
| TAJ | TNFRSF19 | 0 | 0 |
| TCCR | IL27RA | 0 | 0 |
| TGF-b R II | TGFBR2 | 0 | 0 |
| TIMD3 | HAVCR2 | 0 | 0 |
| TWEAKR | TNFRSF12A | 0 | 0 |
| UNC5H3 | UNC5C | 0 | 0 |
| UNC5H4 | UNC5D | 0 | 0 |
| PDE7A | PDE7A | 0 | 0 |
| AMPK a1b1g1 | PRKAA1 PRKAB1 | 0 | 0 |
| | PRKAG1 | | |
| K-ras | KRAS | 0 | 0 |
| NMT1 | NMT1 | 0 | 0 |
| PDE9A | PDE9A | 0 | 1 |
| PPID | PPID | 0 | 0 |
| PSME3 | PSME3 | 0 | 0 |
| GCKR | GCKR | 0 | 0 |
| CK2-A1:B | CSNK2A1 CSNK2B | 0 | 0 |
| CK2-A2:B | CSNK2A2 CSNK2B | 0 | 0 |
| PDK1 | PDK1 | 0 | 0 |
| KIF23 | KIF23 | 0 | 0 |
| IMDH1 | IMPDH1 | 0 | 0 |
| HMGR | HMGCR | 0 | 0 |
| PCSK9 | PCSK9 | 0 | 0 |
| NR1D1 | NR1D1 | 0 | 0 |
| PPIE | PPIE | 0 | 0 |
| MP2K4 | MAP2K4 | 0 | 0 |
| JNK2 | MAPK9 | 0 | 0 |
| AMPK a2b2g1 | PRKAA2 PRKAB2 | 0 | 0 |
| | PRKAG1 | | |
| cGMP-stimulated PDE | PDE2A | 0 | 1 |
| Cyclophilin F | PPIF | 0 | 0 |
| DRAK2 | STK17B | 0 | 0 |
| IMDH2 | IMPDH2 | 0 | 0 |
| PDE11 | PDE11A | 0 | 0 |
| PDE3A | PDE3A | 0 | 0 |
| PDE4D | PDE4D | 0 | 1 |
| PDE5A | PDE5A | 0 | 0 |
| TAK1-TAB1 | MAP3K7 TAB1 | 0 | 0 |
| TYK2 | TYK2 | 0 | 1 |
| ABL2 | ABL2 | 0 | 1 |
| BCAR3 | BCAR3 | 0 | 1 |
| calreticulin | CALR | 0 | 1 |
| GRB2-related adapter | GRAP2 | 0 | 1 |
| protein 2 | | | |
| MMP-16 | MMP16 | 0 | 1 |
| SHC1 | SHC1 | 0 | 0 |
| GHC2 | SLC25A18 | 0 | 1 |
| Eotaxin | CCL11 | 0 | 0 |
| Coagulation Factor IXab | F9 | 0 | 0 |
| Elastase | ELANE | 0 | 0 |
| Apo E2 | APOE | 0 | 1 |
| Prothrombin | F2 | 0 | 1 |
| CD70 | CD70 | 0 | 0 |
| annexin VI | ANXA6 | 0 | 0 |
| B7-2 | CD86 | 0 | 0 |
| calgranulin B | S100A9 | 0 | 0 |
| Caspase-10 | CASP10 | 0 | 0 |
| CBPE | CPE | 0 | 0 |
| CKAP2 | CKAP2 | 0 | 1 |
| CPNE1 | CPNE1 | 0 | 0 |
| Cyclin B1 | CCNB1 | 0 | 0 |
| DLL1 | DLL1 | 0 | 0 |
| hnRNP A2/B1 | HNRNPA2B1 | 0 | 0 |
| HVEM | TNFRSF14 | 0 | 0 |
| Keratin 18 | KRT18 | 0 | 0 |
| LIGHT | TNFSF14 | 0 | 0 |
| MIF | MIF | 0 | 0 |
| NLGNX | NLGN4X | 0 | 0 |
| OMD | OMD | 0 | 0 |
| PIM1 | PIM1 | 0 | 1 |
| Semaphorin 3E | SEMA3E | 0 | 0 |
| SET | SET | 0 | 0 |
| BAFF Receptor | TNFRSF13C | 0 | 1 |
| BRF-1 | BRF1 | 0 | 1 |
| sLeptin R | LEPR | 0 | 0 |
| DR6 | TNFRSF21 | 0 | 0 |
| CAD15 | CDH15 | 0 | 1 |
| CD27 | CD27 | 1 | 0 |
| RANK | TNFRSF11A | 0 | 1 |
| ALCAM | ALCAM | 0 | 0 |
| CYTN | CST1 | 0 | 0 |
| IL-17 RC | IL17RC | 0 | 0 |
| PKC-B-II | PRKCB | 0 | 0 |
| PKC-G | PRKCG | 1 | 0 |
| SLAF7 | SLAMF7 | 0 | 0 |
| SRCN1 | SRC | 0 | 0 |
| Stress-induced- | STIP1 | 0 | 0 |
| phosphoprotein 1 | | | |
| Testican-1 | SPOCK1 | 0 | 0 |
| Testican-2 | SPOCK2 | 0 | 0 |
| RUXF | SNRPF | 0 | 0 |

TABLE 4

| | | | +Higher |
|---|---|---|---|
| | | | Concentration in |
| | | | Parkinson's |
| | | | disease |
| | | | −Lower |
| | | P-value | Concentration in |
| | | from | Parkinson's |
| ProteinName | Entrez Name | model | disease |
| IDE | IDE | 3.35E−08 | + |
| PPase | PPA1 | 3.63E−08 | + |
| eIF-5 | EIF5 | 4.99E−08 | + |
| RAN | RAN | 5.30E−08 | + |
| HNRPQ | SYNCRIP | 1.09E−07 | + |
| Cofilin-1 | CFL1 | 2.87E−07 | + |
| CK2-A1:B | CSNK2A1 | 5.16E−07 | + |
| | CSNK2B | | |
| FGF7 | FGF7 | 5.72E−07 | + |
| eIF-5A-1 | EIF5A | 5.82E−07 | + |
| C1r | C1R | 6.30E−07 | − |
| ERK-1 | MAPK3 | 8.66E−07 | + |
| Transketolase | TKT | 1.63E−06 | + |
| GAPDH liver | GAPDH | 2.27E−06 | + |
| Cyclophilin A | PPIA | 2.27E−06 | + |
| PA2G4 | PA2G4 | 2.45E−06 | + |
| SRCN1 | SRC | 3.31E−06 | + |
| Peroxiredoxin-6 | PRDX6 | 3.82E−06 | + |
| AREG | AREG | 4.06E−06 | + |
| DRG-1 | VTA1 | 4.13E−06 | + |
| MMP-10 | MMP10 | 4.48E−06 | − |
| Caspase-3 | CASP3 | 4.64E−06 | + |
| JAM-C | JAM3 | 4.93E−06 | + |
| AMPM2 | METAP2 | 5.63E−06 | + |
| Sphingosine kinase 1 | SPHK1 | 5.81E−06 | + |
| Ubiquitin + 1 | RPS27A | 6.04E−06 | + |
| RAC1 | RAC1 | 8.13E−06 | + |
| GSK-3 alpha/beta | GSK3A GSK3B | 8.52E−06 | + |
| Growth hormone | GHR | 1.01E−05 | − |
| receptor | | | |
| PLPP | PDXP | 1.07E−05 | + |
| UBE2N | UBE2N | 1.34E−05 | + |
| Sorting nexin 4 | SNX4 | 1.41E−05 | + |
| PDE5A | PDE5A | 1.47E−05 | + |
| Rab GDP dissociation | GDI2 | 1.56E−05 | + |
| inhibitor beta | | | |
| KPCI | PRKCI | 1.64E−05 | + |
| NDP kinase B | NME2 | 1.64E−05 | + |
| PAFAH beta subunit | PAFAH1B2 | 2.02E−05 | + |
| Myokinase human | AK1 | 2.09E−05 | + |
| Triosephosphate | TPI1 | 2.11E−05 | + |
| isomerase | | | |
| Carbonic anhydrase I | CA1 | 2.49E−05 | + |
| MDHC | MDH1 | 3.00E−05 | + |
| SNAA | NAPA | 3.02E−05 | + |
| IL-2 sRg | IL2RG | 3.13E−05 | + |
| MAPK2 | MAPKAPK2 | 3.16E−05 | + |
| LKHA4 | LTA4H | 3.30E−05 | − |
| IMB1 | KPNB1 | 4.32E−05 | + |
| Stress-induced- | STIP1 | 4.51E−05 | + |
| phosphoprotein 1 | | | |
| FER | FER | 4.66E−05 | + |
| Aflatoxin B1 aldehyde | AKR7A2 | 5.38E−05 | + |
| reductase | | | |
| PCNA | PCNA | 5.48E−05 | − |
| SBDS | SBDS | 5.73E−05 | + |
| UFC1 | UFC1 | 6.16E−05 | + |
| FYN | FYN | 6.27E−05 | + |
| PPID | PPID | 6.31E−05 | + |
| PKC-A | PRKCA | 8.38E−05 | + |
| Peroxiredoxin-1 | PRDX1 | 8.51E−05 | + |
| IFN-g | IFNG | 1.09E−04 | + |
| Aminoacylase-1 | ACY1 | 1.11E−04 | − |
| M2-PK | PKM2 | 1.15E−04 | + |
| PKC-B-II | PRKCB | 1.25E−04 | + |
| LYNB | LYN | 1.49E−04 | + |
| EF-1-beta | EEF1B2 | 1.79E−04 | + |
| MBD4 | MBD4 | 1.80E−04 | − |
| BTK | BTK | 1.80E−04 | + |
| SHP-2 | PTPN11 | 2.01E−04 | + |
| TCTP | TPT1 | 2.04E−04 | + |
| UFM1 | UFM1 | 2.13E−04 | + |
| AMPK a2b2g1 | PRKAA2 | 2.20E−04 | + |
| | PRKAB2 | | |
| | PRKAG1 | | |
| UBC9 | UBE2I | 2.60E−04 | + |
| Calpain I | CAPN1 CAPNS1 | 2.60E−04 | + |
| Catalase | CAT | 2.80E−04 | + |
| PDPK1 | PDPK1 | 2.84E−04 | + |
| CSK | CSK | 3.13E−04 | + |
| Protease nexin I | SERPINE2 | 3.21E−04 | − |
| PPIE | PPIE | 3.70E−04 | + |
| 41 | EPB41 | 3.75E−04 | + |
| Glutathione S- | GSTP1 | 3.81E−04 | + |
| transferase Pi | | | |
| NSF1C | NSFL1C | 4.17E−04 | + |
| 6-Phosphogluconate | PGD | 4.23E−04 | + |
| dehydrogenase | | | |
| ING1 | ING1 | 4.33E−04 | + |
| SGTA | SGTA | 4.40E−04 | + |
| tau | MAPT | 4.57E−04 | − |
| GDF-9 | GDF9 | 5.36E−04 | − |
| BARK1 | ADRBK1 | 5.50E−04 | + |
| NCC27 | CLIC1 | 6.68E−04 | + |
| BDNF | BDNF | 7.22E−04 | − |
| ULBP-3 | ULBP3 | 7.26E−04 | + |
| 14-3-3 | YWHAB | 7.46E−04 | + |
| | YWHAE | | |
| | YWHAG | | |
| | YWHAH WHAQ | | |
| | YWHAZ SFN | | |
| Plasmin | PLG | 8.61E−04 | − |
| AIP | AIP | 9.26E−04 | + |
| MK01 | MAPK1 | 9.80E−04 | + |

TABLE 5

| UNIPROT | |
|---|---|
| Accession | Protein Name |
| P06241 | FYN oncogene related to SRC, FGR, YES |
| P25098 | adrenergic, beta, receptor kinase 1 |
| P41240 | c-src tyrosine kinase |
| P68400 | casein kinase 2, alpha 1 polypeptide pseudogene; |
| | casein kinase 2, alpha 1 polypeptide |
| P27361 | hypothetical LOC100271831; mitogen-activated |
| | protein kinase 3 |
| P28482 | mitogen-activated protein kinase 1 |
| P31946, Q04917, | 14-3-3 protein family |
| P61981, P27348, | |
| P63104 | |
| P12931 | v-src sarcoma (Schmidt-Ruppin A-2) viral oncogene |
| | homolog |
| P07948 | v-yes-1 Yamaguchi sarcoma viral related oncogene |
| | homolog |

TABLE 6

| Patient Details | Value |
|---|---|
| Number of Patients | 80 |
| Sex | 47 Males, 33 Females |
| Median Age ± SD | 64.83 ± 9.45 |
| Clinical Diagnosis | 12 AD, 11 ALS, 13 FTD, 20 NC, 20 PD, |
| | 4 PSP |

The disclosures of each and every patent, patent application, and publication cited herein are hereby incorporated herein by reference in their entirety.

While the invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method of identifying a subject suspected of having Parkinson's disease for treatment thereof, comprising
a. determining the test level of a set of biomarkers in a sample obtained from the subject;
b. calculating the probability of the subject having Parkinson's disease according to Equation (I);
$$ln \frac{PD}{1 - PD} = A 1 - A 2 ( BDNF ) - A 3 ( Aminoacylase 1 ) - A 4 ( C 1 r ) + A 5 ( RAN ) + A 6 ( SRCN 1 ) + A 7 ( BSP ) + A 8 ( OMD ) - A 9 ( Growth hormone receptor ) - A 10 ( log Age ) - A 11 ( Gender ) ; PD = probability of the subject having Parkinson ' s disease \qquad ( I )$$
wherein when the calculated probability is more than 0.5, then the subject is diagnosed with Parkinson's disease;
wherein the determining is conducted by a method selected from the group consisting of an antibody based assay, ELISA, western blotting, mass spectrometry, micro array, protein microarray, flow cytometry, immunofluorescence, PCR, aptamer-based assay, immunohistochemistry, and a multiplex detection assay;
wherein the set of biomarkers comprises at least brain-derived neurotrophic factor (BDNF), aminoacylase-1, complement component 1 r subcomponent (Cir), Ras-related nuclear protein (RAN), SRC kinase signaling inhibitor 1 (SRCN1), bone sialoprotein (BSP), osteomodulin (OMD), and growth hormone receptor;
wherein when the subject has Parkinson's disease the subject is administered at least one therapeutic compound selected from the group consisting of carbidopa-levodopa, a dopamine agonist, an MAO-B inhibitor, a catechol O-methyltransferase (COMT) inhibitor, an anticholinergic, and amantadine.

2. The method of claim 1, wherein in Equation (I): A1=77.1531; A2=15.1212; A3=3.1383; A4=9.2111; A5=3.1337; A6=6.8268; A7=14.0165; A8=0.2854; A9=15.2483; A10=16.9700; and A11=1.8442.

3. The method of claim 1, wherein the biological sample is a plasma sample.

4. The method of claim 1, wherein the test level of the set of biomarkers is assessed in an aptamer-based assay.

5. The method of claim 1, wherein the test level of the set of biomarkers is assessed using an ELISA.

6. The method of claim 1, wherein the test level of the set of biomarkers is determined by immunoassay.

7. A non-transitory computer readable medium containing computer-readable program code including instructions for performing the method of claim 1.

8. A system for diagnosing a subject having Parkinson's disease comprising:
a) an assay determining the test level of a set of biomarkers;
b) a computer hardware;
c) a software program stored in computer-readable media extracting the test level from the assay; calculating the probability of the subject having Parkinson's disease according to Equation (I) and outputting the result whether the subject having Parkinson's disease.

9. The system of claim 8, wherein the set of biomarkers comprises at least brain-derived neurotrophic factor (BDNF), aminoacylase-1, complement component 1 r subcomponent (C1r), Ras-related nuclear protein (RAN), SRC kinase signaling inhibitor 1 (SRCN1), bone sialoprotein (BSP), osteomodulin (OMD), and growth hormone receptor.

10. The system of claim 8, wherein in Equation (I): A1=77.1531; A2=15.1212; A3=3.1383; A4=9.2111; A5=3.1337; A6=6.8268; A7=14.0165; A8=0.2854; A9=15.2483; A10=16.9700; and A11=1.8442.

11. A kit for diagnosis of Parkinson's disease, the kit comprising testing reagents for a set of biomarker and an instructional material for use thereof, wherein said set of biomarkers comprises at least brain-derived neurotrophic factor (BDNF), aminoacylase-1, complement component 1 r subcomponent (C1r), Ras-related nuclear protein (RAN), SRC kinase signaling inhibitor 1 (SRCN1), bone sialoprotein (BSP); osteomodulin (OMD); and growth hormone receptor.
